(19) United States
(12) Patent Application Publication
(10) Pub. No.: US 2008/0269849 A1
(43) Pub. Date: Oct. 30, 2008

(54) TEMPORAL CONTROL IN PHOTOTHERAPY

(75) Inventor: Charles Alan Lewis, Carrabelle, NY (US)

Correspondence Address: CARTER, DELUCA, FARRELL & SCHMIDT, LLP, 445 BROAD HOLLOW ROAD, SUITE 225, MELVILLE, NY, 11747, US

(73) Assignee: MERGENET MEDICAL, INC., Coconut Creek, FL (US)

(21) Appl. No.: 12/106,885

(22) Filed: Apr. 21, 2008

Related U.S. Application Data
(60) Provisional application No. 60/925,240, filed on Apr. 19, 2007.

(51) Int. Cl. A61B 18/18

(52) U.S. Cl. 607/91

(57) ABSTRACT

An apparatus for delivering phototherapy includes at least one substrate, at least one emitter mounted on the substrate, and which emits at least two peak wavelengths of light, and an electronic circuit that controls emitter timing. The apparatus is configured as a dressing. A corresponding method includes delivering a first pulse of light to the target tissue from the emitter with a peak wavelength of light, and delivering at least a second pulse of light having a peak wavelength of light that is different from the peak wavelength of the first pulse of light, and the steps define a method of delivering a series of pulse sets of light, and the first and second pulses of light define a pulse set of light. Also disclosed are modular phototherapy units, control of timing of phototherapy by a perfusion detector, and use of long wavelength light for hyperbilirubinemia.

### CROSS-REFERENCE TO RELATED APPLICATIONS

This application claims benefit and priority of U.S. Provisional Patent Application Serial U.S. 60/925,240 filed Apr. 19, 2007, the entire contents of which is hereby incorporated by reference herein.

### BACKGROUND

1. Technical Field

The present relates to the field of phototherapy (PT), which is the use of electromagnetic radiation (EMR) in the near UV, visible and near IR ranges for therapeutic effect.

2. Discussion of Related Art

Light is a form of energy which has obvious effects on plants and animals. Light energy is converted to chemical energy in plants by chlorophyll. Light too has effects on animal physiology, beyond its role in vision. Some of these effects are detrimental, such as damage to the skin and eyes from UV light, while other effects are beneficial, such as the production of Vitamin D in the skin.

Phototherapy (PT) has been shown to increase microcirculation, decrease inflammation, promote angiogenesis, and decrease pain. Phototherapy has also been shown to decrease time for wound healing, and improve diabetic neuropathy.

The therapeutic use of light dates back over several thousand years. In India use of black seeds containing psoralen compounds were used with sunlight to treat non-pigmented lesions around 1500 B.C., and Hippocrates is said to have prescribed heliotherapy (sunlight therapy) around 400 BC. More recently, Niels Finsen was awarded the Nobel Prize in medicine for his 1893 discovery that ultraviolet radiation was beneficial in treatment of cutaneous tuberculosis. Throughout much of the 20^{th} century UV light was used in an updated version of the ancient treatment for psoriasis, using artificial light. In 1981 Parrish published an action spectrum for UV light for treatment of psoriasis, and found that wavelengths around 310 nm was most effective for treatment with less tissue injury. UV light is often used in photodynamic therapy, where a chemical agent is used along with light.

Treatment of infants with neonatal jaundice was discovered serendipitously by an observant nursing sister in a clinic in the U.K. in 1957. The nun reported that the infants with jaundice in the sunlight areas of the nursery had their jaundice fade, while those in other areas did not.

About 50% of full term newborns, and a higher percentage of preterm infants have sufficient hyperbilirubinemia to cause mild jaundice. Neonatal hyperbilirubinemia is mostly unconjugated (unbound to albumin), and thus is free to pass the blood brain barrier. This may cause a form of brain damage called kernicterus, where there is deposition of bilirubin in the basal ganglia and brain stem nuclei. Newborns with health difficulties are at higher risks for multiple reasons, not feeding, lower serum albumin, and use of medications which compete for albumin binding.

Phototherapy is an established treatment for hyperbilirubinemia in neonates, and has greatly reduced the need for exchange transfusions. Phototherapy produces configurational photoisomerization and photo-oxidation of bilirubin in the skin and subcutaneous tissues which makes the bilirubin more water soluble. This prevents the bilirubin from crossing the blood brain barrier and causes it to be excreted more rapidly.

Phototherapy is also used in other areas of medicine. Lasers designed for use to cut and destroy tissue were later found to improve healing when used as dispersed light applied to the tissue. Phototherapy with light in the red and near infrared (R/NIR) portion of the spectrum (about 600 to 1400 nanometers (nm)) regions has gained much interest for therapeutic use. (Light from about 590 to 610 nm is amber/orange in color, but for simplicity herein visible light longer than 590 nm will be referred to as red light.)

A wide range of disorders of biological tissue, or their symptoms, have been treated with R/NIR phototherapy, including but not limited to acute and chronic musculoskeletal conditions such as arthritis, back and joint pain, tendonitis, muscle pain, stiffness and myofascial pain. R/NIR Phototherapy is also used to treat such conditions as post surgical complications such as swelling, inflammation, scarring and stiffness; acute trauma, chronic post-traumatic conditions in the soft tissues, bones including sprains, strains, wounds, neurological and neuromuscular conditions, ulcers including infected or non-infected chronic ulcers of different etiology such as venous ulcers, diabetic ulcers, decubitus ulcers, and pressure sores. Red/IR phototherapy is reported to reduce wrinkles, and other signs of aging of the skin. It has been shown to increase microcirculation, decrease inflammation, help diabetic neuropathy, promote angiogenesis, and decrease time for wound healing. It has recently been used for treatment of memory loss.

R/NIR phototherapy is able to affect deeper tissue levels than the UV therapy which is used for superficial skin disorders. The skin is made up of several distinct layers. Light must traverse these layers to reach the target molecules. UV light does not penetrate deeply enough to be effective in treating deep tissue, and carries risks. UV light is mostly absorbed by the outer layers of the epidermis and therefore has limited effect on deep tissue. UV light is also known to cause DNA damage, skin aging, burn injuries and to impair immune function.

The epidermis is made up of 5 layers. The Stratum Basale contains stem cells which can continuously multiply and produce kerotinocytes, as well as containing melanocytes. The next layer is the stratum spinosa which contains 8 to 10 layers of kerotinocytes which phagocytize melanin granules from projections from the melanocytes in the stratum basale. Next are three to five layers of stratum granulosum cells which are aging kerotinocytes, the stratum lucidum which is a thinner layer of dead cells with droplets of eledin which is eventually transformed to keratin, and the outer layers is the stratum corneum which is made up of 20-30 layers of flat dead cells filled with keratin. Melanin, a UV absorbing molecule, is taken up by the kerotinocytes and functions to protect the underlying tissue from UV light, thus preventing most of the UV, as well as short wave visible light (blue and green light) from crossing the epidermis.

Below the epidermis is the dermis which is composed of connective tissue containing collagen and elastic fibers. There are sparse cells in the dermis, which include fibroblasts, macrophages and fat cells. Blood vessels, nerve endings, sweat glands and hair follicles are embedded in the dermis.

R/NIR can penetrate into the dermis, and thus can have an effect on this tissue. It is theorized that R/NIR therapy stimulates fibroblast in the dermis to produce substances involved with healing and growth. Specifically it is thought that much of the action of the light is caused by its effects on the electron carriers of the electron transport chain in the inner membrane of the mitochondria of these cells.

Wound healing may be described as having four component processes.

1. Inflammation increases blood flow to the area, increased delivery of white blood cells which phagocytize microbes and mesenchymal cells which develop into fibroblasts. Blood clotting helps unite the wound.
2. In the Migration component epithelial cells migrate below a scab and cover the wound area. Fibroblasts migrate along fibrin threads and begin the synthesis of collagen and glycoproteins. During this phase damaged blood vessels begin to regenerate.
3. During the Proliferative component there is further growth of the epithelial cells and deposition of collagen fibers and continued growth of blood vessels.
4. In the final Maturation phase the scab disconnects and the collagen fibers become more organized, fibroblasts decrease in number and the blood vessels return to normal.

The specific wavelengths commonly used in R/NIR phototherapy are those for which commercial medical lasers were already available, for example He—Ne laser (lambda=632.8 nm), rather than finding the wavelengths corresponding to the target molecules. In recent years, Karu has tested the stimulation of DNA and RNA synthesis rate and cell adhesion when exposed to monochromatic light sources. She found several active regions for phototherapy in the R/NIR range. These active regions have peaks around 620, 680, 760, and 820 nm. Currently phototherapy is used in short sessions typically lasting only several minutes, and repeated in later days. This results in inconvenience and increased therapy costs.

Blue or white light is typically used for treatment of hyperbilirubinemia. Fluorescent lights, halogen lams, and sun light have been used. It was originally thought that UV light was necessary for treating neonatal jaundice. This is incorrect and dangerous for infants. The infant lens is very transparent to blue and UV light, and the retina is susceptible to damage from light especially below 450 nm. The risk of retinal injury is even greater when the infant is given oxygen therapy. There is even concern that the UV light from the mercury bands in normal fluorescent lights poses risk to neonates receiving oxygen therapy.

### SUMMARY

In view of the foregoing description of the current status of the field of phototherapy, the present disclosure advances the state of the art of phototherapy by temporal control in phototherapy. In particular, the present disclosure relates to an apparatus for delivering phototherapy that includes at least one substrate configured to enable mounting at least one light emitter; at least one emitter mounted on the at least one substrate, and that is capable of emitting at least two peak wavelengths of light; and an electronic circuit configured to control the timing of emission of the at least one emitter. The electronic circuit is in electronic communication with the at least one emitter and the apparatus is configured as a dressing for optical communication enabling irradiation of a target tissue.

In one embodiment, the one, or more than one, light emitter is a laser emitter and/or a light emitting diode. The electronic circuit may include at least one processor and the one, or more than one, processor is configured to control temporal sequencing of emission of light by the one, or more than one, emitter at least two different peak wavelengths.

In one embodiment of the phototherapy apparatus, the one, or more than one, emitter is configured with at least one of the following: (1) a blue emitter and one of a phosphor and a scintillator, the phosphor and the scintillator emitting light at least one peak wavelength longer than 500 nanometers (nm); (2) a light emitting diode emitting white light; and (3) at least one light emitter that is configured to deliver a pulse of irradiation to the tissue with a duration of less than a second.

In another embodiment of the phototherapy apparatus, the one, or more than one, substrate includes at least a first and a second substrate; and the one, or more than one, emitter includes at least a first emitter mounted on the first substrate and at least a second emitter mounted on the second substrate, wherein the first substrate and the at least first emitter define a first modular phototherapy apparatus and the second substrate and the at least second emitter define a second modular phototherapy apparatus, and wherein the first and second modular phototherapy apparatuses are at least one of the following: (1) physically connected to at least one another; (2) mounted at least one of on and within a common structure; and (3) in electric communication with each other.

In another embodiment of the phototherapy apparatus, the phototherapy apparatus further includes at least one power source, wherein the one, or more than one, power source is configured to provide power to the apparatus to effect the emission of the light, and wherein the one, or more than one, power source is at least one of the following: (1) a power source having sufficient capacity to power the emitters for at least one hour; (2) a battery; and (3) a power source configured for ambulatory use. In one embodiment, the phototherapy apparatus further includes a reflective surface that is configured to reflect light from the one, or more than one, emitter towards the target tissue.

In still another embodiment, at least a portion of the phototherapy apparatus is configured to be wearable by a subject, and the at least a portion that is wearable is configured as a piece of apparel and/or a bandage. In one embodiment, phototherapy apparatus further includes a translucent dressing that is configured wherein the light of the one, or more than one, emitter is directed through the translucent dressing.

In yet another embodiment, the phototherapy apparatus further includes at least one of the following: at least one light sensor configured to detect data samples of light passing through the target tissue and of changes in the light passing through the target tissue; and at least one processor wherein the processor is: (1) capable of determining from the data samples, and with respect to a subject, at least one of the timing of the pulse, the pulse pressure, and the respiratory cycle, and with respect to at least one of a subject and a target tissue, determining from the data samples at least one of the oxygen saturation of the blood and the hemoglobin content of the blood; and (2) capable of timing the delivery of phototherapy according to a predetermined phase of the pulse cycle.

In another embodiment of the phototherapy apparatus, with respect to the one, or more than one, the one, or more than one, processor at least one of:

(1) is configured to control the one, or more than one, emitter to deliver at least a first pulse and at least a second pulse of light for irradiation to the target tissue;

(2) is configured to control the one, or more than one, emitter to repeat the at least first pulse of light and at least second pulse of light as pulse sets; and

(3) is configured to create a delay between pulse sets; and/or with respect to the one, or more than one, emitter at least one of:

(1) is a blue emitter with a wavelength ranging between about 450 and about 500 nanometers (nm) and wherein light emitted from the blue emitter is delivered with a first pulse, and includes at least one second emitter with a wavelength ranging between about 500 and about 700 nanometers (nm) and wherein light emitted from the at least one second emitter is delivered with a second pulse in the pulse set; and

(2) is an emitter with a wavelength ranging between about 800 and about 900 nanometers (nm) and wherein light emitted from the at least one emitter is delivered with a first pulse, and includes at least one second emitter with a wavelength ranging between about 600 and about 700 nanometers (nm) and wherein light emitted from the at least one second emitter is delivered with a second pulse in the pulse set.

In another embodiment, the phototherapy apparatus includes at least one light source configured to deliver phototherapy with a peak wavelength between 580 and 1350 nm; at least one light sensor configured to detect light passing through the target tissue and changes in the light passing through the target tissue and at least one processor configured to at least one of: (1) measure changes in at least one of blood volume and light absorption of the blood passing through the target tissue; (2) enable correlation with respect to the subject of the changes in the light passing through the target tissue with at least one of the timing of the pulse, the pulse pressure, the oxygen saturation of the blood, the hemoglobin content of the blood, and the respiratory cycle; and (3) control timing of delivery of phototherapy according a portion of the pulse cycle.
The present disclosure relates also to a method of applying phototherapy to a subject, and includes the steps of: providing at least one light emitter; delivering a first pulse of light to the target tissue of a subject from the one, or more than one, emitter with a peak wavelength of light; and delivering at least a second pulse of light to the target tissue of the subject from the one, or more than one, emitter wherein the one, or more than one, emitter provides at least one peak wavelength of light that is different from the peak wavelength of the first pulse of light, wherein the steps of delivering a first pulse of light and of delivering a second pulse of light define a method of delivering a series of pulse sets of light, and wherein the first pulse of light and the second pulse of light define a pulse set of light. The method may be implemented wherein the one, or more than one, emitter is at least one of a light emitting diode and a laser.

In one embodiment, the method may further include wherein at least one of: (1) the step of delivering of the at least second pulse of light occurs at a time period of less than about one second after the step of delivering the first pulse of light; (2) the step of delivering of the at least a second pulse set of light occurs at a time period of less than about one minute after the step of delivering the first set of pulses; (3) the step of delivering the at least a second pulse set occurs at a time period of less than about one second after the step of delivering the first set of pulses; and (4) wherein the method of delivering a series of pulse sets of light includes the step of delivering at least three pulse sets over a time period greater than about one hour.

In another embodiment, the method may include wherein at least one of: (1) the first pulse of light in the pulse set has a peak wavelength between about 450 nanometers and about 500 nanometers; (2) the at least a second pulse of light has a peak wavelength between about 500 to about 700 nanometers (nm); (3) the at least a second pulse of light has a peak wavelength between about 565 to about 700 nm; and (4) the phototherapy is applied for treatment of at least one of hyperbilirubinemia, jaundice, hematoma and bruising.

In still another embodiment, the method is implemented wherein at least one of:

(1) wherein the pulse set includes at least: a first pulse of light having a peak wavelength between about 800 nanometers and about 900 nanometers, and wherein the at least second pulse of light has at least one peak wavelength between about 600 nanometers and about 700 nanometers; and (2) wherein the method of applying phototherapy is applied for treatment of at least one of injury, tissue degeneration, tissue discoloration, and hair loss.

The present disclosure relates also to a method for phototherapy for a subject that includes the steps of: providing at least one light source emitting light with a peak wavelength less than about 500 nanometers; providing at least a second light source emitting light with at least one peak wavelength ranging between about 565 nanometers and about 700 nanometers (nm); delivering the light with a peak wavelength of less than about 500 nanometers to tissue of the subject for a time period of greater than about one hour; and at least partially concurrently delivering the light with a peak wavelength ranging between about 565 and about 700 nanometers for a time period of greater than about one hour. In one embodiment, the method for phototherapy for a subject may be implemented wherein the phototherapy is for a subject with at least one of hyperbilirubinemia, jaundice, hematoma and bruising.

The present disclosure relates also to a method of timing delivery of phototherapy to a subject that includes the steps of: measuring at least one phase of the circulatory cycle of the subject; identifying a desired phase of the at least one phase of the circulatory cycle of the subject wherein the desired phase is beneficial for delivery of phototherapy to the subject; and delivering phototherapy to the subject during at least a portion of the desired phase of the circulatory cycle of the subject. The method may be implemented wherein the desired phase of the circulatory cycle is determined by detecting a variance in transmission of light through tissue of the subject.

Additionally, the present disclosure relates also to a method of phototherapy treatment that includes the steps of: providing at least one light emitter, and irradiating target tissue of a subject with light from the one, or more than one, emitter for sufficient time to give therapeutic effect, wherein the one, or more than one, emitter is at least one of:

(1) a blue emitter emitting light with a peak wavelength less than 500 nanometers (nm), the light emitted from the blue emitter including at least a portion of the light irradiating the tissue from the one, or more than one, emitter, the blue emitter coupled with one of a phosphor and a scintillator, the phosphor and the scintillator emitting light at least one peak wavelength longer than 500 nanometers (nm), the light from the one of a phosphor and a scintillator being at least a portion of the light irradiating the tissue from the one, or more than one, emitter; (2) a light emitting diode emitting white light, the white light emitted from the light emitting diode being at least a portion of the light irradiating the tissue from the at least one emitter; (3) configured to emit light cycling in intensity at a rate of at least one cycle per second, the light from the one, or more than one, emitter cycling in intensity being at least a portion of the light irradiating the tissue from the one, or more than one, emitter; and (4) configured to emit light delivering a pulse of irradiation with a duration of less than one second, the light from the one, or more than one, emitter delivering a pulse of irradiation with a duration of less than one second being at least a portion of the light irradiating the tissue from the at least one emitter. The method may be implemented wherein the subject has at least one of hyperbilirubinemia and jaundice.

It is understood that light energy can cause changes in the chemical activity or structural conformation of certain molecules. The present disclosure teaches that sequential application of light at specific wavelengths can drive the conformational changes or the energetic changes in the desired direction making phototherapy more efficient in certain applications. The present disclosure also teaches that in certain applications sustained phototherapy has advantages over the current practice of short term phototherapy which is usually applied by a physician or therapist in a medical setting and lasting only a few minutes per session. The present disclosure also teaches the use of sustained pulsed phototherapy which may be applied for hours or days, and may be worn as apparel or applied to an area of the body as a fixture, bandage or appliance.

The present disclosure teaches the use of timed and sequenced narrow spectrum EMR for improving the beneficial effects of phototherapy, as well as to improve efficiency and reduce risk. It also teaches the use of timing the delivery of phototherapy to the certain parts of the circulatory pulse cycle, and a method of integrating plethysmography and/or pulse oximetry into the phototherapy apparatus. Several embodiments of the present disclosure include single use and wearable PT apparatuses. Also disclosed is the uses of sustained red/NIR phototherapy lasting several hours or longer that is made possible with wearable phototherapy apparatuses.

The present disclosure further teaches that long wave visible light may be used in the photo-conversion of bilirubin to lumirubin.

For purposes of this disclosure and claims, the term light is not limited to visible light but rather includes electromagnetic radiation including the visible, ultra violet and infrared spectrum.

### BRIEF DESCRIPTION OF THE DRAWINGS

Reference will now be made to the accompanying drawing figures, which are not necessarily drawn to scale:

FIG. 1 is a graph showing the absorbance of light according to wavelength of the most important light absorbing substances in the dermis and epidermis;

FIG. 2 is a diagrammatic chart showing the timing for a phototherapy according to a particular embodiment of the present disclosure utilizing emitters with two different wavelengths;

FIG. 3 is a diagrammatic chart showing the timing for a phototherapy according to a particular embodiment of the present disclosure with three different wavelength emitters;

FIG. 4A is a view of the emitter face of a phototherapy unit according to a particular embodiment of the present disclosure;

FIG. 4B is a view of the electrical connection side of a phototherapy unit according to a particular embodiment of the present disclosure;

FIG. 4C is a side view perspective of a phototherapy unit according to a particular embodiment of the present disclosure;

FIG. 4D illustrates two views of a battery compartment which may be incorporated into a particular embodiment of the present disclosure;

FIG. 4E illustrates the upper face of a miniature phototherapy unit according to one embodiment of the present disclosure using laser diodes;

FIG. 5 is a chart showing the wavelengths of light considered most important in low level laser therapy;

FIG. 6A shows the absorption spectrum and emission spectrum of bilirubin.

FIG. 6B shows the action spectrum for ZZ bilirubin and for EZ bilirubin after filtering through human skin;

FIG. 7A shows a view of another configuration of a phototherapy module according to a particular embodiment of the present disclosure on a side having phototherapy modules disposed thereon;

FIG. 7B illustrates a pulse oximetry probe according to prior art, which may be used in conjunction with certain embodiments of the present disclosure;

FIG. 7C shows another side of the configuration of a phototherapy module according to FIG. 7A;

FIG. 7D is an illustration of a method for branching connectors for wiring together a plurality of phototherapy modules or units according to a particular embodiment of the present disclosure;

FIG. 8 shows a photoplethysmographic waveform used for timing of delivery of phototherapy under a particular embodiment of this present disclosure;

FIG. 9 is a view of an array of phototherapy modules working in conjunction with a pulse-oximeter according to a particular embodiment of the present disclosure and configured within a bandage that is applied to a leg of a subject;

FIG. 10A is a view of phototherapy units or modules placed in a wound dressing material according to a particular embodiment of the present disclosure;

FIG. 10B is a view of a wound dressing material conformation for use with phototherapy units or modules according to a particular embodiment of the present disclosure;

FIG. 11A illustrates a phototherapy apparatus configured into a booty according to a particular embodiment of the present disclosure;

FIG. 11B illustrates the phototherapy apparatus of FIG. 11A configured into a booty according to another particular embodiment of the present disclosure;

FIG. 12A illustrates one embodiment of a piece of apparel configured as a mask and containing phototherapy modules or units according to the present disclosure;

FIG. 12B illustrates another embodiment of a piece of apparel configured as a mask and containing phototherapy modules or units according to the present disclosure;

FIG. 13 illustrates a phototherapy apparatus configured into a baseball cap according to one embodiment of the present disclosure;

FIG. 14A is a view of phototherapy modules made on flexible printed circuits connected together into to grid or tile format according to a particular embodiment of the present disclosure;

FIG. 14B is a more detailed view of a phototherapy module made on flexible on printed circuit according to a particular embodiment of the present disclosure;

FIG. 15 show additional embodiments of phototherapy modules which may be made on flexible printed circuit material according to particular embodiments of the present disclosure; and

FIGS. 16A, through 16F. show the spectral output of various lights:

FIG. 16A illustrates the spectral output of a “Special Blue” fluorescent lamp used in the treatment of hyperbilirubinemia;

FIG. 16B shows the spectral output of a white fluorescent lamp;

FIG. 16C illustrates the spectral output of a helium neon laser;

FIG. 16D illustrates the spectral output of a blue L.E.D. with a peak wavelength of 465 nm;

FIG. 16E illustrates one spectral output of a white L.E.D.; and

FIG. 16F illustrates another spectral output of a white L.E.D.

### DETAILED DESCRIPTION

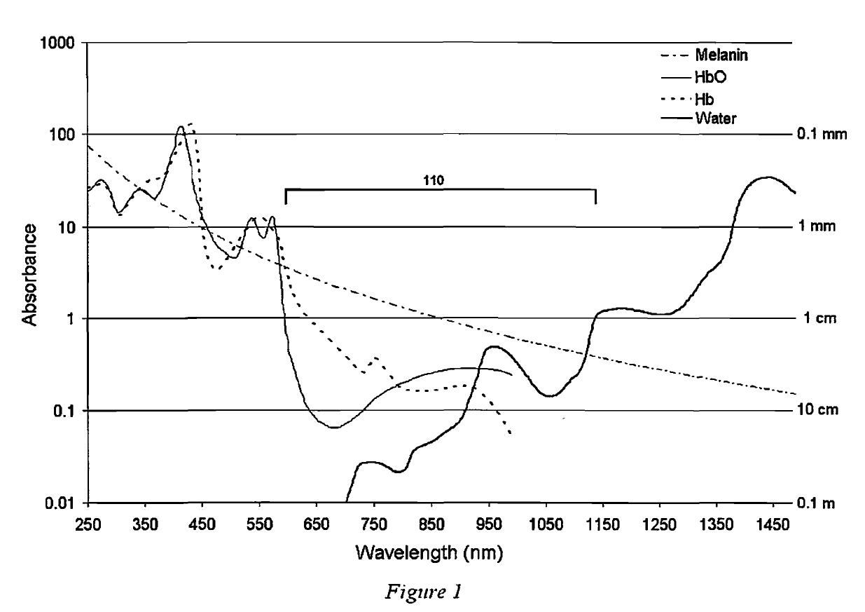

Phototherapy requires that light be transmitted to the target molecules in the body, but non-target substances in the skin and underlying tissue (which absorb light) prevent it from reaching the target molecules. FIG. 1 shows the major light absorbers in the cutaneous tissue, and the window for light penetration through this tissue. These four major absorbers are reduced hemoglobin (Hb), oxyhemoglobin (HbO), melanin and water. The vertical axis on the left shows typical absorbance of light at different wavelengths (horizontal axis) for these substances. Absorbance is equal to −log₁₀(I/Io) where I is the intensity of light that has passed through an absorber and Io is the intensity of the light before it has passed through the absorber.

The vertical axis on the right shows the relative penetration depth for light for different wavelengths. For phototherapy to have biological affect, light must penetrate to the target tissue.

In FIG. 1 the absorption of light by hemoglobin (thin solid line) and oxyhemoglobin (dashed line) are illustrated. The oxygenation state of hemoglobin affects its absorbance characteristics. It can be seen in this illustration that at about 660 nm and at 1000 nm that there is a large difference in absorption depending on the oxygen saturation state of hemoglobin. Water (heavy solid line) is a very good absorber of electromagnetic radiation, but has a window in the near UV to the near IR range. Absorption of light by water falls to its lowest point at about 400 nm.

The sum of hemoglobin (saturated or unsaturated), melanin and water absorbance gives an estimate of the penetration of light. Bracket 110 indicates an area between about 580 nm and 1125 nm where light most easily gives sufficient penetration for therapeutic exposure of the tissue. The areas between 460 and 580 and between 1125 and 1320 allow lesser transmission of light, but still may also be used for phototherapy The optical window, between about 580 nm to about 1320 nm herein referred to as the red/near infrared (R/NIR) window, is used in phototherapy for healing.

Hemoglobin's absorbance depends on its oxygenation state, as well as the amount of hemoglobin present. Thus, a person with anemia would have less interference for light passage. An area of poor perfusion would tend to have a higher percentage of reduced hemoglobin (Hb), and light transmission would be affected by this. Tissues such as cartilage have little blood supply and may transmit light better.

Melanin absorbs heavily in the UV and blue area of the spectrum, and plays an important role in protecting the underlying dermis from UV radiation. The layer of melanin is very thin. There is as much as a 10 fold difference in melanin content between individuals, with Caucasians having a lower content than most other racial groups. There is also a wide variance within individuals depending on sun exposure which promotes production of melanin. Disrupted skin, wounds and depigmented lesions may not have melanin deposits that block light, as well the palms and soles are less pigmented.

### Sequential Phototherapy for Hyperbilirubinemia

Use of alternating or sequential targeted wavelength light to induce or impede molecular reactions in a desired direction can be thought of as “optical pumping”. The present disclosure teaches the concept of optical pumping for phototherapy. One example of optical pumping with clinical utility is its use in the photo-conversion of bilirubin.

The conversions of Bilirubin to Lumirubin is a two step process. The present disclosure teaches the method of inducing this process by sequential irradiation of bilirubin with two or more targeted narrow spectrum light emitters in order get a more efficient conversion of bilirubin to lumirubin.

Bilirubin is hydrophobic and lipophylic, allowing it to cross the blood brain barrier where it can damage the brain. Photo-oxidation of bilirubin changes it into a more hydrophilic form. Light exposure causes bilirubin to undergo structural isomerization and photo-oxidation. Hyperbilirubinemia is treated with light in infants because light changes the bilirubin from a water-insoluble form to a water soluble form which prevents it from crossing the blood brain barrier and makes it filterable by the kidneys and thus allows it to be eliminated, principally into the urine.

When photo-isomerized bilirubin becomes Lumirubin (EZ-cyclobilirubin), the water soluble form. The photoconversion reaction bilirubin is as follows:

4Z, 15E Bilirubin [symbol] 4Z, 15Z Bilirubin [symbol] 4E, 15Z Bilirubin→15Z Lumirubin,

and where simplified nomenclature is used:

ZE Bilirubin [symbol] ZZ Bilirubin [symbol] EZ Bilirubin→Lumirubin.

ZZ Bilirubin refers to native bilirubin, ZE bilirubin is also referred to as photobilirubin, and lumirubin is also known as cyclobilirubin. The first step occurs when ZZ bilirubin is photoisomerized to either the ZE or the EZ form. This isomerization is reversible, and the reaction generally greatly favors the formation of the ZE form. The photo-oxidation of EZ bilirubin is irreversible, and forms lumirubin which is water soluble and can be eliminated through the kidneys.

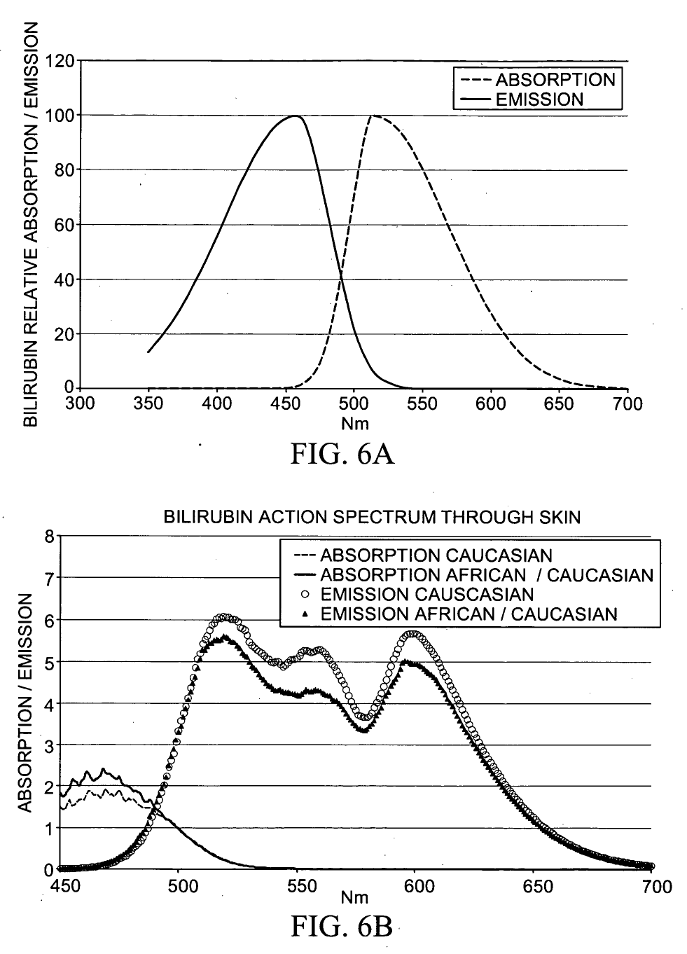

FIG. 6A is a graph illustrating the absorption spectrum for ZZ bilirubin on the left as a solid line and the emission spectrum for photo-excited bilirubin on the right as a dashed line. Bilirubin absorbs light best at about 451 nm. In vivo, bilirubin is bound to albumin, and this shifts the peak absorption of bilirubin to about 458 nm. This wave length correlates to the color blue, although ZZ bilirubin does absorb light in the UVA range as well. It is at this wave length (about 458 nm) which irradiation most efficiently excites the geometric photoisomerization from (ZZ)-bilirubin bound to human serum albumin to ZE and EZ bilirubin.

FIG. 6A also shows the emission spectrum for bilirubin. The present disclosure teaches the concept of counter-irradiation against emission fluorescence, in which light is delivered to a molecule at its emission spectrum in order to promote a further reaction. Thus, irradiation of EZ bilirubin with light at its emission spectrum promotes the photo-oxidation reaction which converts EZ bilirubin to lumirubin.

This stepped conversion of bilirubin is an example of optical pumping, where a first reaction (irradiation of ZZ bilirubin at its absorption spectrum) primes a second reaction (the conversion of ZE bilirubin to lumirubin) Thus, the present disclosure teaches the method of sequential phototherapy also referred to herein as optical pumping. In the present example sequential exposure of two or more narrow spectrum wavelength light sources can be used for the efficient photoisomerization of bilirubin to lumirubin. The first narrow spectrum emitter in the blue range, followed by longer wavelength light, for the second irreversible step from photoisomerized bilirubin to lumirubin.

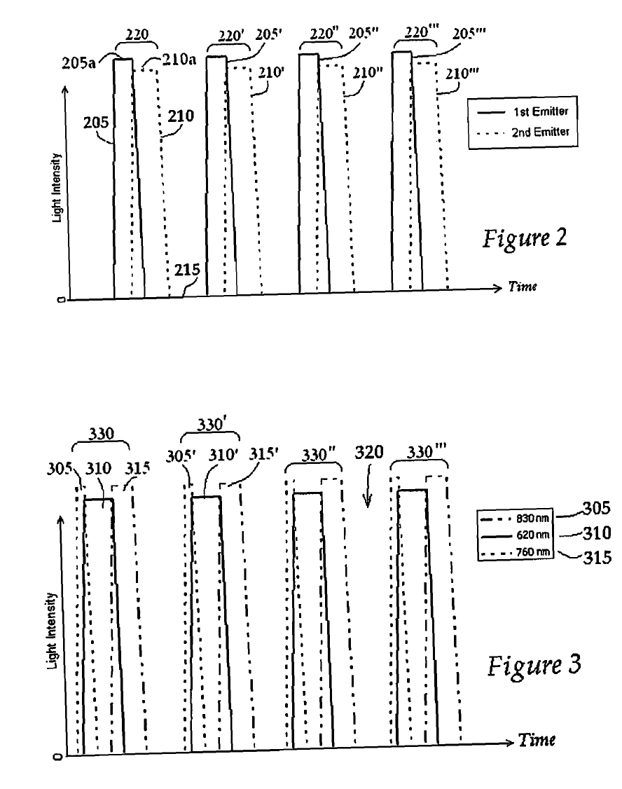

The present disclosure further teaches cycling of narrow wavelength emitters for phototherapy. FIGS. 2 and 3 illustrate this sequential timing. Time is shown along the horizontal axis. The vertical axis illustrates light intensity with zero intensity (the emitter off) at baseline and a peak value yielding light at an intensity sufficient to cause the desired effect. The height along the vertical axis in FIG. 2 and FIG. 3 are not to scale and heights vary only for clarity. A plateau time is shown, but is not meant to indicate scale or relationship of plateau duration. In another embodiment of the present disclosure for phototherapy the light intensity of one or more or the emitters may cycle without the intensity falling to zero.

In one embodiment of the present disclosure, a narrow spectrum light source with a peak output at or near the absorption maxima for ZZ bilirubin may be used for the first narrow spectrum emitter of electromagnetic radiation (EMR), followed by a second emitter selected for stimulating the conversion of EZ bilirubin to lumirubin.

In the case of phototherapy for hyperbilirubinemia, the first light source 205 thus causes the first conformational change in bilirubin, and the second light source 210 causes an irreversible second change to lumirubin. This sequential pulse set may be repeated immediately, or there may be a delay as before the cycle is repeated. FIG. 2 illustrates an example of this cycling. In prior art phototherapy the molecular substrate is receives radiation in random order, and there is less probability that the photo-conversion of bilirubin to be driven in the desired direction. Additionally, when not sequenced, more energy is required, and there is more exposure to blue light.

Zietz et al (2004) found that the decay of bilirubin fluorescence is very rapid, peaking after about 250 to 1000 femtoseconds. During this stimulated emission, there is an excited state absorption at a peak wavelength of about 515 nm.

The second photo-induced transformation of EZ bilirubin occurs about 150 to 2000 femtoseconds after the first transformation. Femtosecond lasers may be quick enough to time this reaction, but it is simpler to overlap the timing with more easily accessible L.E.D. emitters. Thus overlapping L.E.D. output of various wavelengths will give the desired sequential illumination. Some commonly used divisions of time are: Femtosecond=10⁻¹⁵ seconds, Picosecond=10⁻¹² seconds, Nanosecond=10⁻⁹ seconds, Microsecond (μs)=10⁻⁶ seconds, and Millisecond=10⁻³ seconds.

Since it may be costly or impractical at present to use high speed narrow spectrum light sources, such as femtosecond lasers which can switch this quickly, narrow spectrum light sources which overlap sequentially may be used.

L.E.D. emitters may be used for phototherapy. However the time it takes for an L.E.D. to turn on and off may not be a quick as the time it takes for sequential photo-induced biological reaction to occur. A typical L.E.D. may have for example, a rise time of 40 ns, a pulse of 10 μs, and a fall time of 60 ns. This rise and fall time are indicated in FIG. 2 and FIG. 3 by the upslope and down-slope of intensity axis, where it may be seen that the bases of the pulses are shown wider than the plateaus. The plateau may represent the pulse time of the L.E.D., or the time the emitter is at its plateau level.

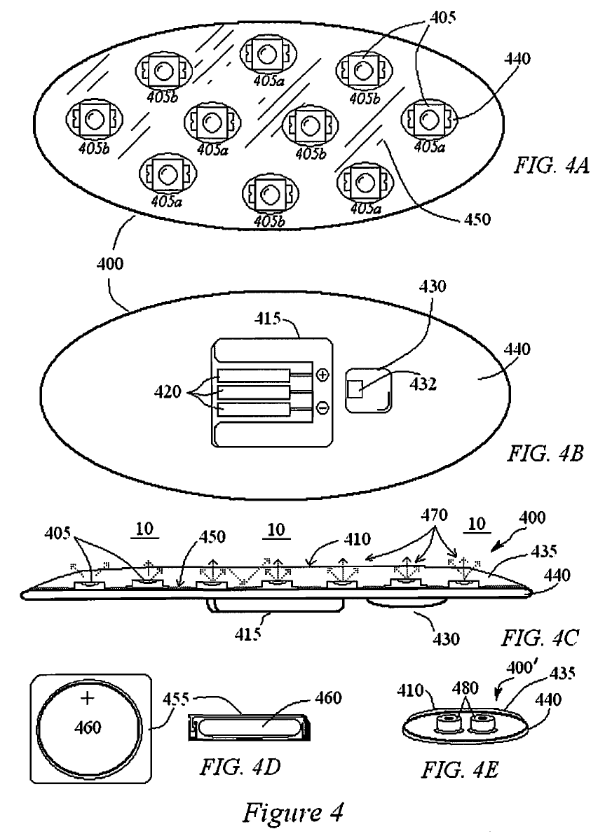

As illustrated in FIG. 2, the application of the phototherapy is sequenced. The first light source 205 gives a brief pulse of light, followed by the second light source 210. These emitters may correspond for example to the L.E.D.s 405a and 405b in FIG. 4A.

In the embodiment illustrated by FIG. 2 emitters are timed so that the second emitter 210 does not start until after the first light source 205 has reached its plateau output 205a, and the emitter may remain illuminated long enough for the second source 210 to reach its plateau output 210a, where upon the first light source could be turned off as illustrated in FIG. 2. A microprocessor, such as microprocessor 430 controls the timing of the emitters. The on time of the emitters is shown as being similar, however this is not necessarily the case, and depending on the substrate for phototherapy the time lengths may vary.

In this illustration the plateau phases of the two emitters overlap, however this present disclosure is not limited to use of overlapping pulse phases for sequential pulse phototherapy. The illustration also shows an off time 215 following the two emitter pulses. The pair or set of pulses by the emitters forms a pulse set. This pulse set is shown to repeatedly cycle with an off time after each set. In other embodiments of this present disclosure one or more emitters of a certain peak wavelength may remain illuminated while one or more emitters of a different peak wavelength cycle. In one embodiment the pulse time for emitters in this present disclosure may be as slow as one minute. In another embodiment very rapid cycling well under a second is utilized, and many cycles of the pulse sets may be delivered per second.

Thus, in view of FIG. 2, the present disclosure teaches a method of applying phototherapy to a subject which includes the steps of:

providing at least one light emitter; delivering a first pulse 205 of light to the target tissue of a subject from the one or more emitters with a peak wavelength of light; and

delivering at least a second pulse 210 of light to the target tissue of the subject from the one or more emitter wherein the one or more emitters provides at least one peak wavelength of light 210 that is different from the peak wavelength 205 of the first pulse of light 205. The steps of delivering a first pulse of light 205 and of delivering a second pulse of light 210 define a method of delivering a series of pulse sets 220 of light, and

the first pulse of light 205 and the second pulse of light 210 define a pulse set of light 220. The one or more emitters may be a light emitting diode and/or a laser.

Furthermore, the present disclosure teaches a method of phototherapy wherein at least one of the following occurs:

(1) the step of delivering of the at least second pulse of light 210 occurs at a time period of less than about one second after the step of delivering the first pulse of light 205;

(2) the step of delivering of the at least a second pulse set 220′ of light occurs at a time period of less than about one minute after the step of delivering the first set of pulses 220;

(3) the step of delivering the at least a second pulse set 220′ occurs at a time period of less than about one second after the step of delivering the first set of pulses 220; and

(4) wherein the method of delivering a series of pulse sets of light 220, 220′, 220″, 220″′ etc. includes the step of delivering at least three pulse sets 220, 220′ and 220″ over a time period greater than about one hour.

Continuing to refer to FIG. 2, the present disclosure teaches a method of applying phototherapy to a subject, wherein at least one of the following occurs:

(1) the first pulse of light 205 in the pulse set 220 has a peak wavelength between about 450 nanometers and about 500 nanometers;

(2) the one or more second pulses of light 210′, 210″, 210″ etc. has a peak wavelength between about 500 to about 700 nanometers (nm);

(3) the one or more second pulses of light 210′, 210″, 210″ etc. has a peak wavelength between about 565 to about 700 nm; and

(4) the phototherapy is applied for treatment of at least one of hyperbilirubinemia, jaundice, hematoma and bruising.

The emitters may cycle without any off time, however there is typically advantage to having an off time. These advantages include more efficient use of power saving and less heat build up in the tissue. There may also be therapeutic benefits. This method may be used with the various embodiments of this disclosure.

Cycling the L.E.D.s allows delivery of more EMR energy with less risk of tissue damage, lowers risk of heat injury to the patient, and saves energy, which may be important for battery operated and wearable apparatuses. Using specific wavelengths of light which target the absorption maxima for the target molecules also greatly increases the efficiency of the phototherapy and lowers the EMR required.

Since the molecular reactions in phototherapy occur in response to specific wavelengths of light, it is desirable to use emitters specifically targeted to these reactions. For example, broad spectrum white light is often used in phototherapy for hyperbilirubinemia. However, much of the light does not specifically target the bilirubin molecule, and thus much higher light intensities are required for this use. The subject of this therapy is often a premature infant, who is thus exposed to light and heat which may have undesirable effects including increased body temperature, dehydration and photo-damage to the skin and eyes. Further this non-specific light uses large amounts of electrical energy and creates waste heat. Heat and the energy consumption of such lights make them impractical for use as portable devices or for use in close proximity to the skin.

The use of narrow spectral output emitters, such as lasers and light emitting diodes (L.E.D.s), can allow for the specific targeting of the molecular reactions which respond to phototherapy by selecting emitters with peak spectral outputs at the target wavelengths for the photo-reaction. By selecting narrow band emitters with a peak wavelength output which matches the wavelengths for the maximum photoreaction, phototherapy can be much more efficient than with use of broad spectrum emitters.

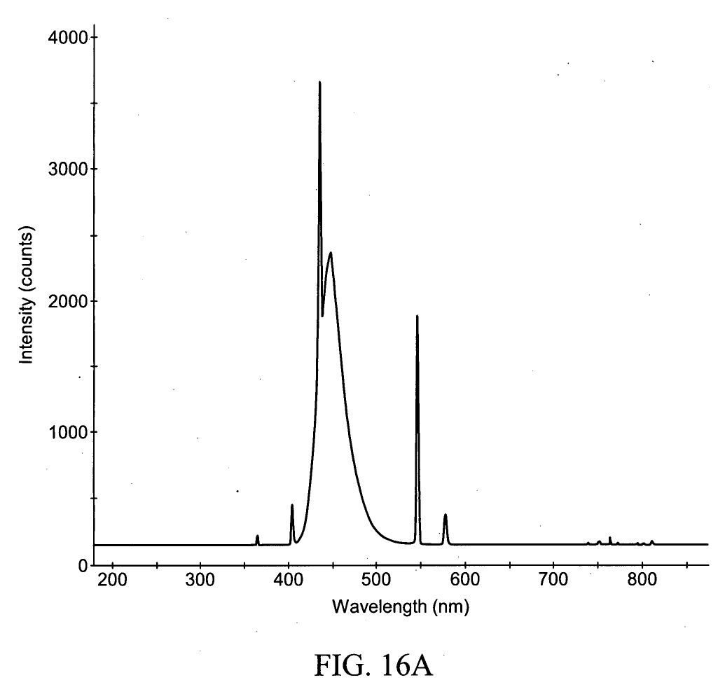

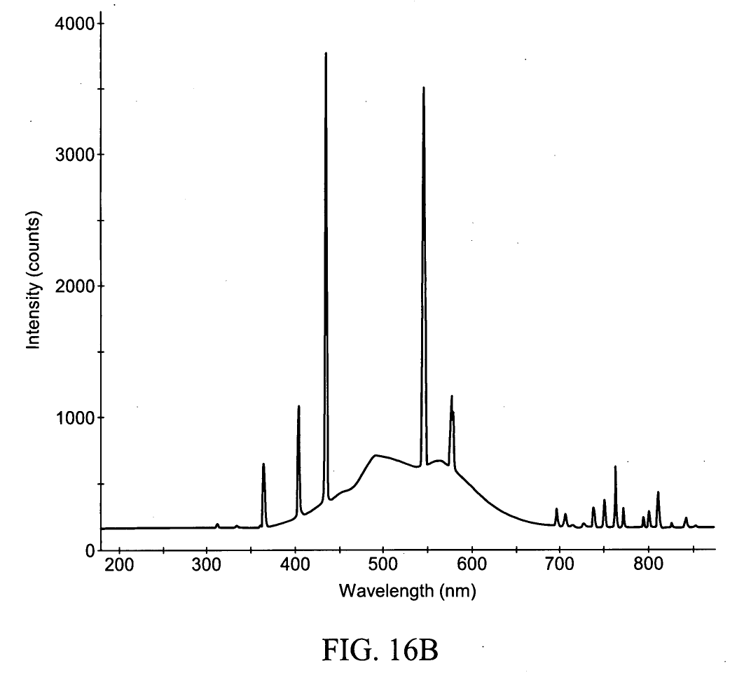

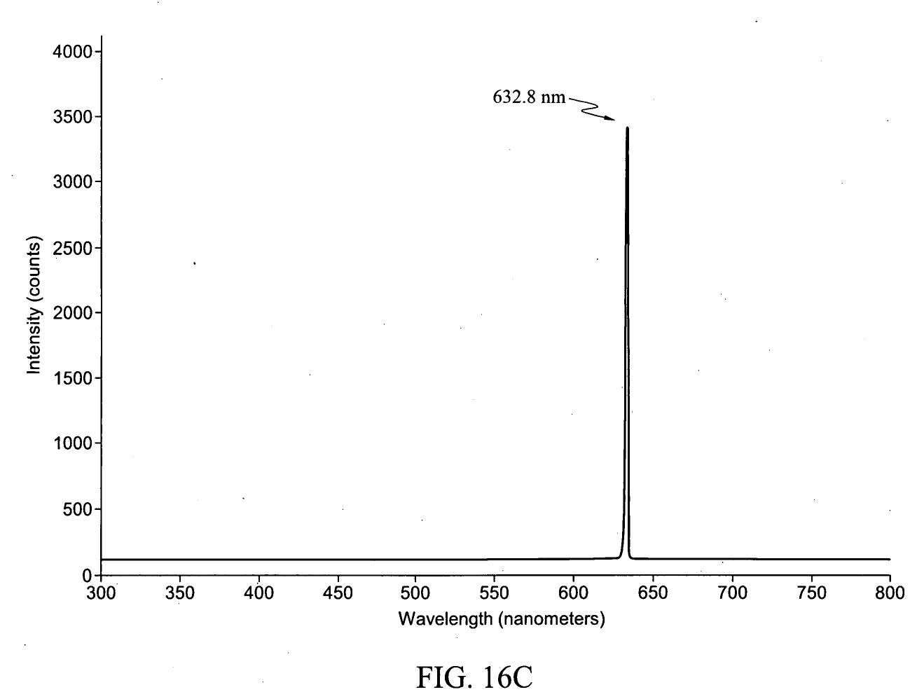

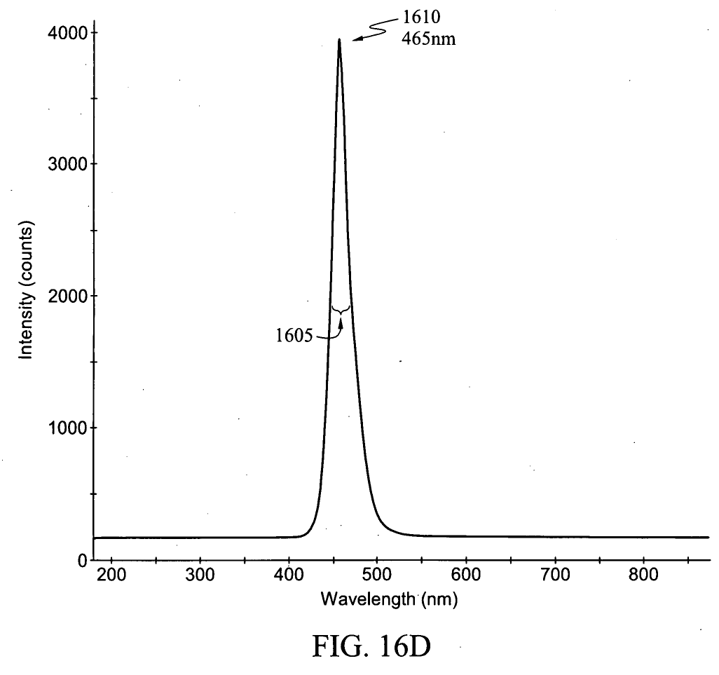

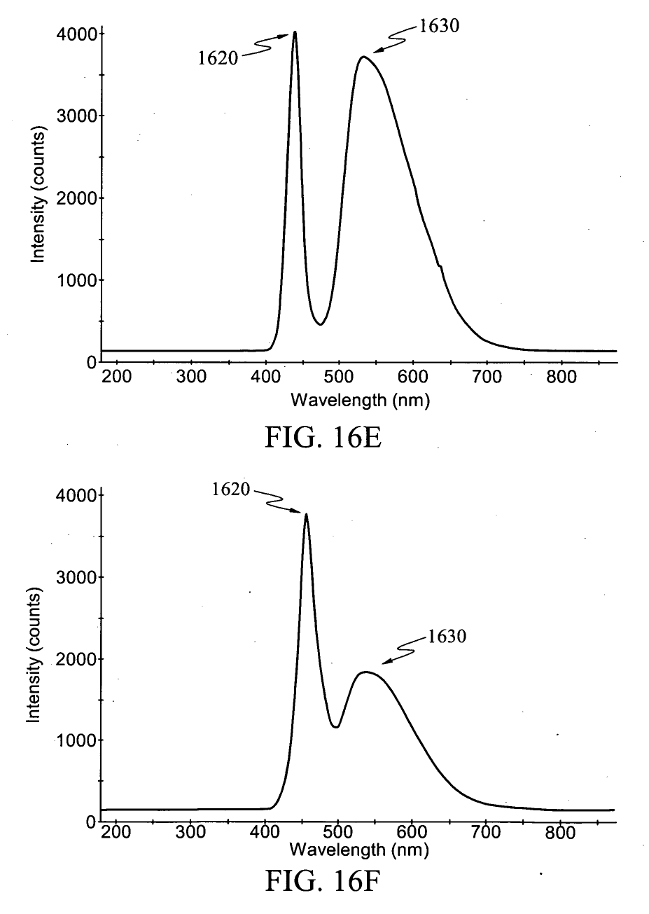

In one embodiment of the present disclosure small diode lasers may be used for phototherapy. An example of such diode laser is the Sanyo DL-8142-201 830 nm, infrared wavelength laser diode which is supplied in a 5.6 mm housing. Even smaller package sizes are expected to be commercially available in the near future. Lasers emit light narrowly around a single wavelength, with a full width half maximum (FWHM) of a few nm to well under one nm. This is illustrated in FIG. 16C. The light emitted by an L.E.D. is emitted in a range of wavelengths centered around a peak wavelength. FIG. 16D illustrates the FWHM 1605 for a blue L.E.D. with a peak spectral output 1610 of about 465 nm. The FWHM tends to widen with increasing wavelength so that near IR L.E.D.s typically exhibit a FWHM of 60 nm or more. The FWHM range also depends on the type of L.E.D. This range is called the spectral width of the emitter. The spectral width of L.E.D.s is usually well adapted for application for phototherapy. Among other factors, one should select the proper peak wavelength to match the photoreaction.

Sequential R/NIR Phototherapy

The R/NIR window 110 between 580 nm and 1125 nm, and less so the area up to about 1325 nm, is important in the application of phototherapy for healing and other therapeutic effects as it allows EMR in the red and near infrared range to reach the target molecules. Red/Near Infrared light passing through this optical window is thought by many scientists in the field to exert action by its effect on electron transport in the mitochondrial proton pump. The inner mitochondrial membrane contains five (5) complexes of integral membrane proteins which are involved in the electron transport chain; NADH dehydrogenase, succinate dehydrogenase, cytochrome c reductase, cytochrome c oxidase, ATP synthase as well as two freely diffusible molecules; ubiquinone and cytochrome c that shuttle electrons. These contain metallic atoms; iron copper, zinc, magnesium, and it is thought that these metallic complexes may be the targets for phototherapy. In particular it is the copper atoms in cytochrome c oxidase that are thought to participate in the beneficial effects of phototherapy.

Time resolved spectroscopy of cytochrome C oxidase reveals that in its resting state it absorbs light at about 830 nm. It is thought that light stimulates electron transfer in cytochrome C oxidase. After this it then absorbs light at 606 nm and at 430 nm for a few nanoseconds. Light at about 430 nm or at 605 nm may cause photolysis of the bonding between cytochrome c, and cytochrome c oxidase. Removal of this oxidized cytochrome c, a soluble heme protein, allows it to disassociate with the cytochrome c oxide complex more quickly, thus leaving the bonding site available to a reduced cytochrome c molecule. Here, the present disclosure teaches another example where the sequential irradiation of tissue by different wavelengths of light act as an optical pump advantageous for phototherapy. Thus, in order to more efficiently photoactivate cytochrome c oxidase the tissue is first exposed to light at about 830 nm as a first step and then by light at about 606 and or 430 nm as a second step. In one embodiment red light at about 606 nm is used for the second step because of the increased transmission of red light through the tissue as compared to blue light at 430 nm.

Karu demonstrated that simultaneous irradiation of cell cultures with two (2) monochromatic sources could decrease DNA synthesis compared to one source, and that depending on the order which the light source were applied would either promote or decrease DNA synthesis. When several minutes of irradiation with light at 760 nm was followed a few minutes later with several minutes of EMR at 633 nm the amount of DNA synthesis was greater, and if first exposing the cells with 633 nm light followed a few minutes later by 760 nm light DNA synthesis over the next several days was decreased.

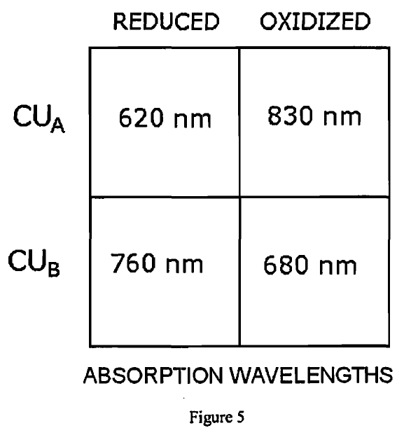

Karu has identified four active areas for phototherapy for red to near infrared light. These are about 613-624 nm, and 667-684 nm in the visible spectra, and two near infrared maxima with peak positions in the ranges of 750-773 nm and 812-846 nm. These correlate well to the four cytochrome c oxidase redox active metal centers; two heme A prosthetic groups (cytochrome a and cytochrome a3) and two copper centers (CuA and CuB). These redox centers absorb light according to their redox state. These four areas are referred to as 620, 680, 760 and 830 herein for simplicity, and are shown in FIG. 5 along with their redox status. When Cu_{A} receives an electron from cytochrome c, it becomes reduced. When it gives up this electron it becomes oxidized. In Cytochrome c Oxidase, the electron transport chain moves electrons in cytochrome c oxidase in a cascade from Cu_{A} to hemeₐ to heme₃ to Cu_{B}. Light energy can affect this cascade. Sequential phototherapy may assist in the electron transfer moving forward through the redox cycle. Other researchers have found another area useful for phototherapy at around 900 nm.

The present disclosure teaches the use of rapid sequential cycling of short pulses of light for the therapeutic phototherapy such as low level laser phototherapy. In one configuration of the invention the tissue is irradiated with a short burst of infrared light at around 830 nm, followed by irradiation with red light at around 620 nm.

While traditional low level laser phototherapy uses lasers, the present disclosure teaches the use of L.E.D. light sources in place of lasers for therapeutic phototherapy. In one embodiment of the present disclosure a pulse of narrow spectrum light such as may be produced with an EMR emitter such as an L.E.D. or laser at about 830 nm is followed by a narrow light spectrum emitter at about 680 nm, as illustrated in FIG. 2.

This method of photoactivation is implemented by sequential irradiation from at least two different emitters, as illustrated in FIG. 2, which shows emittance cycling. In this diagram emitters 205 and 210 have rapid rise times, plateaus, and rapid fall times. Emitter 205 is turned on and followed by emitter 210 forming a pulse set 220. There may be a relaxation time 215 before the next pulse set 220 cycle of irradiation. Also, more than one emitter color may be used at the same time. Light intensity falls between pulse sets 220, but are not required to go to zero in this method.

FIG. 3 illustrates the use of three sequential narrow spectrum emitters. Time is shown along the horizontal axis. In this illustration an emitter 305 with a first wavelength is shown as having a peak wavelength at 830 nm and is followed by an emitter with a second wavelength 310 shown as having a peak wavelength at 620 nm, and this followed by a third emitter 315 shown having a peak wavelength at 760 nm, forming a pulse set 330, followed pulse sets 330′, 330″ and 330″′. In a different embodiment of this present disclosure for example, the second and third emitters may pulse simultaneously. Off times 320 are illustrated between pulse sets, but are not required. The wave lengths shown are meant to be exemplary, and are not meant to limit which wavelengths may be used in this method. Similarly, while FIG. 2 and FIG. 3 show two and three sequential emitter wavelengths respectively, these embodiments are not meant to limit the number or sequence of the emitters used under the methods of this disclosure. For example, in another embodiment of this disclosure the first pulse may use more than one narrow spectrum emitter followed by one or more narrow spectrum emitters. Further the duration for the plateau may be different for different emitters depending on the reaction characteristics of the substrate being targeted.

In the method of phototherapy illustrated in FIG. 3, at least one of the following occurs:

(1) the pulse set includes at least:

a first pulse of light 305 having a peak wavelength between about 800 nanometers and about 900 nanometers, and

the at least second pulse 310 of light has at least one peak wavelength between about 600 nanometers and about 700 nanometers; and

(2) the method of applying phototherapy is applied for treatment of at least one of injury, tissue degeneration, tissue discoloration, and hair loss.

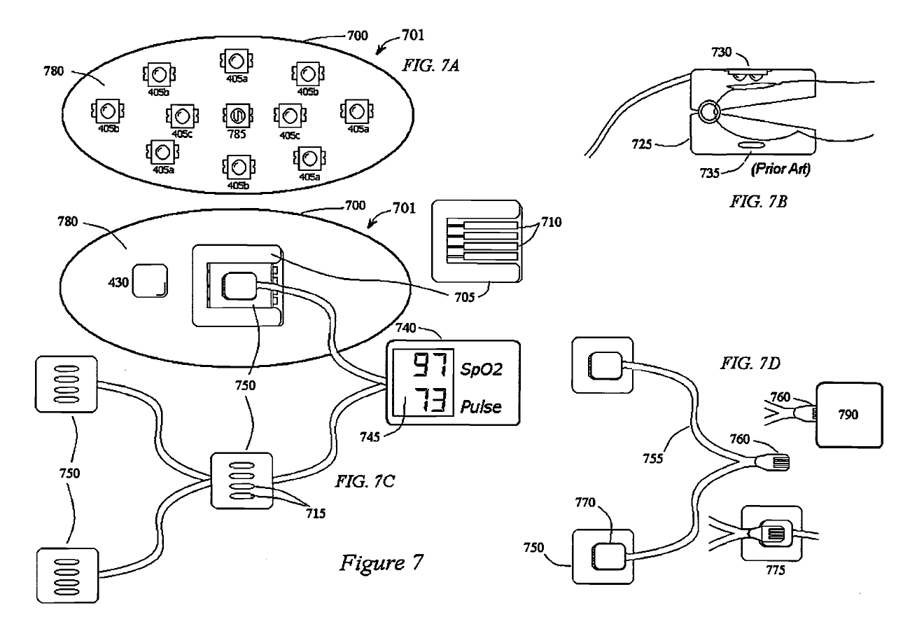

An electronic controller or processor (e.g., a microprocessor) such as 430 as shown in FIG. 4B and FIG. 7C, or housed in 740 in FIG. 7C, implements the timing and sequences of emitters in this method. In one embodiment of this disclosure the controller is able to rapidly sequence the emitters, with ability to control the sequences to fractions of a second. The power supply for apparatus 7C may also be housed in the pulse oximetry unit 740, and may contain batteries for powering the unit and the phototherapy modules in one configuration of this disclosure.

The present disclosure thus teaches the method of rapid sequential irradiation using narrow wavelength emitters for phototherapy.

Referring specifically to FIG. 4 and to FIG. 7 for example, phototherapy apparatus 400 (see FIGS. 4A, 4B, 4C) and 701 (see FIGS. 7A and 7C) may be configured wherein the processor 432 (see FIG. 4B) or the processor housed in pulse oximeter 740 is at least configured to control the one or more emitters 405 (e.g., 405a, 405b, 405c) to deliver at least a first pulse and at least a second pulse of light for irradiation to the target tissue 10 and/or is configured to control the one or more emitters 405 (e.g., 405a, 405b, 405c) to repeat the at least first pulse of light and at least second pulse of light as pulse sets, and/or is configured to create a delay between pulse sets. Additionally phototherapy apparatus 400 and 701 may be configured wherein the one or more emitters 405 (e.g., 405a, 405b, 405c) is at least one of the following: (1) a blue emitter with a wavelength ranging between about 450 and about 500 nanometers (nm) and wherein light emitted from the blue emitter is delivered with a first pulse, and includes at least one second emitter with a wavelength ranging between about 500 and about 700 nanometers (nm) and wherein light emitted from the one or more second emitters is delivered with a second pulse in the pulse set; and/or

(2) an emitter with a wavelength ranging between about 800 and about 900 nanometers (nm) and wherein light emitted from the one or more emitters is delivered with a first pulse, and includes at least one second emitter with a wavelength ranging between about 600 and about 700 nanometers (nm) and wherein light emitted from the one or more second emitters is delivered with a second pulse in the pulse set.

Specific Spectrum for treatment of Hyperbilirubinemia

When bilirubin absorbs light, some of this energy is released in the form of fluorescence. This occurs most efficiently with light at the peak absorption area for bilirubin. Irradiated bilirubin emits light with a peak emissive wave length of about 515 nm as shown by the dashed line in FIG. 6A.

The skin acts as a filter for external light passing though it. FIG. 6B illustrates the adjusted action spectrum for ZZ bilirubin and the emission spectrum for photoisomerized bilirubin after the effect of light absorption by the skin. The action spectra for bilirubin after transmission of light through skin of a Caucasian person and a person of mixed African and European descent are illustrated. Little difference between these individuals is shown in the location of peaks for these action spectrums. For persons with more melanin, the peak of the action spectrum for ZZ bilirubin may move slightly towards longer wavelengths. The vertical axis is on an adjusted scale of percent transmission, as depth of transmission through the tissue was not tightly controlled. The peak in the action spectrum for photo-isomerization of ZZ to EZ bilirubin is on the left as a solid line and the action spectrum for photo-oxidation from EZ bilirubin to lumirubin, as taught herein, is on the right, shown with a dashed line.

A shift in the absorption and emission spectra for ZZ bilirubin can be seen. The most effective wavelengths for photoisomerization for ZZ bilirubin centers at about 470 nm, and this may be slightly greater in persons with high melanin content in their skin. The photo conversion of EZ bilirubin to lumirubin however has a broad range which results from the relative ease which red light transmits through the skin. The dashed line in FIG. 6B shows the action spectrum for photo-oxidation of EZ bilirubin to lumirubin after filtering through the skin to be broad and have peaks at around 520 nm, 560 nm, and 600 nm.

Filtering by the absorbing substances in the tissue also affect light used for R/NIR phototherapy. Since hemoglobin, water and melanin absorb light, the acts as skin slightly skews the effective peak wavelengths for phototherapy, to a slightly longer wavelengths. Thus in one embodiment of Red/NIR phototherapy a first emitter at about 840 nm may be used. Similarly in one embodiment of Red/NIR phototherapy a second emitter at about 620 nm my be used.

It is generally understood that shorter wavelengths of light (ultraviolet, blue and even green light) are more damaging to the tissues, and more likely to cause retinal injury, aptosis and cell death. Light at around 435 nm is near the peak for “blue hazard” light which can damage the retinal pigmented epithelial cells in adults, but because of the increased transmittance of blue light by the lenses of infants, shorter wavelength blue light can be even more damaging to infants than adults. Longer wavelength light is less hazardous.

The present disclosure teaches that light sources for phototherapy should minimize light exposure which is harmful to the eye. There is also less potential for photo-damage to the skin within the longer wavelength range. Standard neonatal phototherapy has been found to be a strong risk factor for pigmented nevus development in childhood, and may be associated with a lifetime increase in risk for malignant melanoma. Phototherapy for hyperbilirubinemia may increase the incidence of retinopathy of prematurity if an eye shield is not used correctly. Use of narrow wavelength phototherapy for treatment of hyperbilirubinemia as described in the present disclosure avoids or reduces this risk.

For photo conversion of bilirubin blue light may be used in constant emission as the first emitter and with the second emitter turning off and on within the present disclosure. However, there is an advantages to embodiments which cycle the blue light, thus limiting exposure to the shorter and more potentially damaging wave lengths. Further it can be seen in FIG. 6B that a wide range of emitters would work for the second emitter or set of emitters. Peak wavelengths from the green to red range may be used. Thus, selection of one or more emitter for the second reaction if bilirubin conversion can be made based on safety of the emitter as well as availability of emitters. The present disclosure teaches the method of using yellow or longer wavelength visible light for conversion of bilirubin to lumirubin. More specifically this refers to the photo-oxidation of EZ bilirubin to lumirubin, and more specifically refers to light from about 565 nm to 700 nm. In more general terms, the present disclosure teaches the use of yellow to red light in the treatment of hyperbilirubinemia. L.E.D. emitters are readily available in this color range, and have less potential for photo-injury compared to shorter wavelength light of the same intensity. In a one embodiment this yellow to red light would be used in concert with blue light to first stimulate the photo-isomerization of ZZ bilirubin. One embodiment of the present disclosure uses sequential irradiation using blue light followed by longer wavelength light.

Referring to FIG. 11A in conjunction with FIG. 4A for example, the present disclosure teaches a method for phototherapy for a subject, and particularly suitable for a subject with hyperbilirubinemia, jaundice, hematoma or bruising, that includes the steps of:

providing at least one light source 405a emitting light with a peak wavelength less than about 500 nanometers;

providing at least a second light source 405b emitting light with at least one peak wavelength ranging between about 565 nanometers and about 700 nanometers (nm);

delivering the light from light source 405a with a peak wavelength of less than about 500 nanometers to tissue 10 of the subject for a time period of greater than about one hour; and

at least partially concurrently delivering the light from light source 405b with a peak wavelength ranging between about 565 and about 700 nanometers for a time period of greater than about one hour.

FIGS. 16E and 16F show the emittance from two different white L.E.D.s.; FIG. 16E having a warmer white light than FIG. 16F. These L.E.D.s are made with a blue L.E.D. emitter 1620 and phosphor or scintillator which when excited by the light from the blue L.E.D. causes the emission of a second longer peak wavelength light output 1630. Comparing the emission spectrum of these white L.E.D.s to the action spectrum for bilirubin in FIG. 6B it can be seen that they match well. Also there is an inherent although very short delay between the onset of the blue emitter and the emission of longer wavelength phosphor or scintillator. Thus by rapidly cycling of this type of white L.E.D., a sequential emission can be created with a blue emitter followed by a longer wavelength emission. Thus we teach that white L.E.D.s may be used for treatment of hyperbilirubinemia. One embodiment of the present invention is the use of a white L.E.D. for phototherapy of hyperbilirubinemia. In this embodiment the L.E.D. may be cycled on and off. This causes the sequential irradiation of blue light, followed by the longer wavelength emission of the phosphor or scintillator. In one embodiment this cycling may be done in excess of hundreds of times a second. In another embodiment this cycling may be done more slowly. When cycling is done quickly enough, the light appears continuous to the human eye. The cycling time used may be influenced by the decay time, which is slower for a phosphor or very rapid for a scintillator.

Referring to FIGS. 4C and 11A-11B, it can be appreciated that the present disclosure describes a method of phototherapy that, while being a general method of phototherapy, is particularly suitable as a method of phototherapy for subjects having hyperbilirubinemia or jaundice. (FIGS. 4A to 4E are discussed in more detail below). However, it can be appreciated that the method includes providing at least one light emitter, e.g., light emitters 405 of phototherapy units 400 or of phototherapy units 700 and irradiating target tissue 10 of a subject with light 470 from the one or more emitters 405 for sufficient time to give therapeutic effect. The one or more emitters 405, and in particular emitters 405a and 405b, may include a blue emitter emitting light with a peak wavelength less than 500 nanometers (nm). The blue emitter may be coupled with a phosphor and/or a scintillator. The phosphor and the scintillator emit light at least one peak wavelength longer than 500 nanometers (nm). The light emitted from the blue emitter may form or be included as at least a portion of the light irradiating the tissue from the one or more emitters 405 while the light from the phosphor and/or the scintillator may also form or be included as at least a portion of the light irradiating the tissue from the one or more emitters 405. Alternatively, the one or more emitters 405 may be a light emitting diode emitting white light. The white light emitted from the light emitting diode may form or be included as at least a portion of the light irradiating the tissue from the one or more emitter 405.

In still another embodiment, the one or more emitters 405 may be configured to emit light 470 cycling in intensity at a rate of at least one cycle per second. The light 470 from the one or more emitters 405 that cycles in intensity may form or be included as at least a portion of the light irradiating the tissue 10 from the one or more emitters 405. In yet another embodiment, the one or more emitters 405 may be configured to emit light 470 delivering a pulse of irradiation with a duration of less than one second. The light 470 from the one or more emitters delivering a pulse of irradiation with a duration of less than one second may form or be included as at least a portion of the light irradiating the tissue 10 from the one or more emitters 405.

Thus it can also be appreciated that in view of the foregoing method of phototherapy treatment of a subject, with reference to FIGS. 4A-4E and 11A-11B, the present disclosure relates to an apparatus 400 for delivering phototherapy, wherein at least one emitter 405 is configured with at a blue emitter and a phosphor and/or a scintillator. The phosphor and the scintillator emit light at least one peak wavelength longer than 500 nanometers (nm). Alternatively, the one or more emitters 405 may be a light emitting diode that emits white light and/or at least one light emitter configured to deliver a pulse of irradiation to the tissue 10 with a duration of less than a second.

Referring now to FIGS. 16D and 16E, in comparing FIGS. 16D and 16E, it can be seen that the relative sizes of these peaks can be controlled during manufacture; a warmer white light can be made where the blue peak is less intense, or a cooler white can be made where the blue peak is more intense than the phosphor/scintillator peak. Thus for use of a white L.E.D.s for phototherapy for hyperbilirubinemia, emitters maybe selected with a balance of a emitter blue and phosphor with a favorable balance for the photo-conversion of bilirubin.

In another embodiment at least one RGB L.E.D. may be used for treatment of hyperbilirubinemia where the blue emitter emission is pulsed followed by the at least one of the green and red emitters.

### Phototherapy and Perfusion Timing

The present disclosure teaches that phototherapy apparatuses may be timed so that the delivery of light occurs when there is less absorption of light by interfering absorbers which vary temporally, such as occurs during the circulatory pulse with hemoglobin. For example, phototherapy pulses for one condition may be delivered during the nadir of perfusion when the arterial capillaries are less distended, when blood movement is slower and when the ratio of arterial to venous blood in the tissue is lower. Thus, phototherapy may be used in concert with a plethysmograph or pulse oximeter to time the delivery of phototherapy during the portion of the pulse most favorable to transmission of the therapeutic wavelengths. Alternatively the phototherapy apparatus according to the present disclosure, for example as illustrated in FIGS. 7C and 11A, may also be integrated with a pulse oximeter or photoplethysmograph.

As shown in FIG. 1, reduced hemoglobin (dotted line) absorbs more light than oxyhemoglobin (thin solid line) from about 590 nm to about 800 nm, and above 850 nm oxyhemoglobin absorbs more light.

A pulse oximeter typically uses two or more narrow spectrum light source such as L.E.D.s to determine the difference in hemoglobin saturation levels. FIG. 7B shows a prior art pulse oximetry probe 725. Two L.E.D.s 730 are typically used, one at 600 to 750 nm (red) and the other from 850 to 1000 nm (infrared). The light from the L.E.D.s transmits through the finger tip and is sensed by the photosensor 735. The L.E.D.s quickly alternate, and a photosensor compares the relative absorption of light to determine the percent oxygen saturation.

Pulse oximeters also detect the pulse through photoplethysmography. With each pulse of the heart there is enough pressure to distend the arteries and arterioles in the skin and subcutaneous tissues. The increased amount of blood is sufficient to decrease light transmission. A small venous plexus pulse may be detected, as well as other physiologic changes including the respiratory cycle. Photoplethysmography may be used to monitor circulation in the area being treated with phototherapy, such as might be important in the treatment of diabetic ulcers of pressure sores.

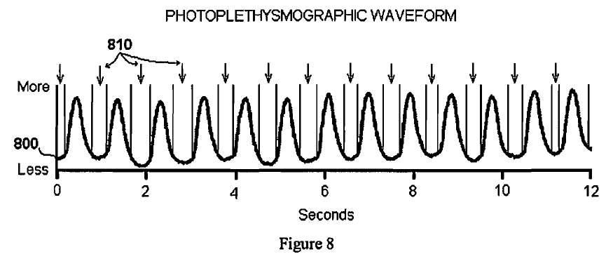

FIG. 8 illustrates a photoplethysmographic waveform 800 (heavy undulating line) from pulse oximetry. The peaks occur when there is more blood in the arterial circulation (systolic phase) and the light transmitted to the photosensor decreases, and the valleys occur when there is less blood in arterial circulation. Arrows 810 show timing during the diastolic phase under one configuration of the present disclosure when it is advantageous for delivery phototherapy during periods when less blood is in the arteries. During this time, there is a higher ratio of venous blood. The venous blood has a slightly lower oxygen saturation.

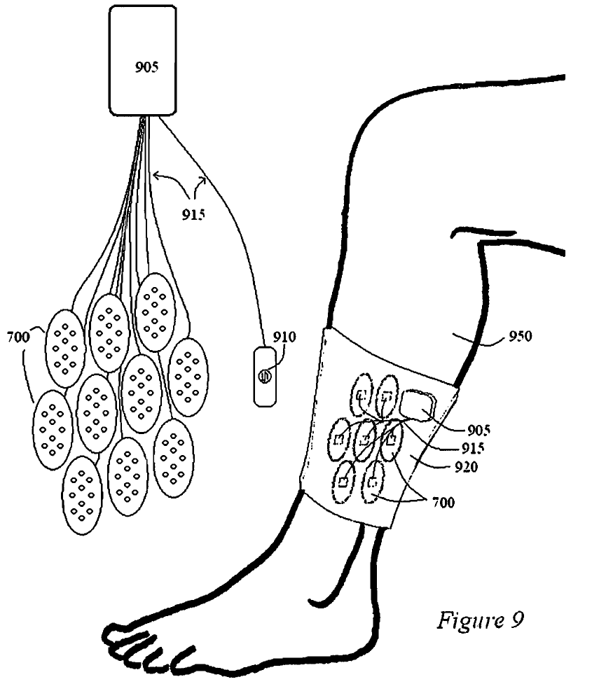

A photoplethysmograph requires only a single light source, the transmission of which is attenuated by the increase in blood flow, while the pulse oximeter requires two different narrow spectrum light sources. FIG. 9 shows a cluster of phototherapy modules 700 integrated with a microprocessor 905 and a photosensor 910. This apparatus allows for detection of the pulse and the control of the phototherapy modules to be used according to the desired portion of the pulse cycle. The photosensor would be placed in to detect light having transmitted through the patient's tissue in order to detect the pulse.

Plethysmographic data for timing of phototherapy pulses can be done even if pulse oximetry is not. Only a single emitter and detector are required. Any of the emitters used for phototherapy between about 580 nm and 1150 nm are adapted to be used to obtain plethysmographic data. The housing for the microprocessor 905 may also house the power supply for the microprocessor and the phototherapy modules. As with other configuration of this disclosure, batteries may be used with this apparatus which allow mobility during phototherapy.

Since the wavelengths used for pulse oximetry are similar to those which may be used in phototherapy, the light from phototherapy may be used for pulse oximetry measures in some applications, or supply at least some of the required wavelengths. An embodiment of this is illustrated as phototherapy apparatus 701 in FIG. 7A and FIG. 7C. In other configurations for phototherapy supplemental emitters may be required. In yet other configurations one or more L.E.D.s may be used as the photosensor. In one example, a phototherapy L.E.D. on one side of the tissue may be used as the emitter some of the time and as a light sensor at other times. Similarly reflective pulse oximetry may use L.E.D.s as photosensors, and may use these L.E.D.s as dual use emitters/detectors.

For treatment of hyperbilirubinemia, photoplethysmographic data would also allow timing of the phototherapy to deliver phototherapy during the nadir of perfusion. It can be seen in FIG. 1 that the notch in hemoglobin absorption between around 450 and 500 nm is deeper for the reduced form of hemoglobin. Thus in a certain embodiment phototherapy for hyperbilirubinemia blue light may timed to deliver light during the diastolic portion of the pulse cycle when hemoglobin is least saturated, and less likely to interfere with the irradiation of bilirubin. In another embodiment according to this disclosure delivery of light for Red/NIR phototherapy may be timed to the first part of the perfusion trough. During this time less arterial blood is present, and the hemoglobin present is more fully oxygenated and thus more transparent to light between 600 and 700 nm.

Referring to FIG. 11A for example, the present disclosure teaches a method of timing delivery of phototherapy to a subject that includes the steps of:

measuring at least one phase of the circulatory cycle of the subject;

identifying a desired phase of the one or more phases of the circulatory cycle of the subject wherein the desired phase is beneficial for delivery of phototherapy to the subject; and

delivering phototherapy to the subject during at least a portion of the desired phase of the circulatory cycle of the subject.

In FIG. 7B a pulse oximetry probe 725 is shown positioned on a finger as per prior art. Two L.E.D. emitters 730 are shown in proximity to the finger nail and a photoreceptor 735 is shown at the pad of the finger. Such a probe may also be used to gather pulse oximetry or plethysmography data in coordination with phototherapy.

FIG. 7C illustrates the apparatus with a pulse oximeter 740. It is shown with a display 745 of oxygen saturation and pulse values, although this is not required for the phototherapeutic role of the apparatus. Photosensor 780 may be used for plethysmography or pulse oximetry under various embodiments of the present disclosure. The pulse oximeter can act here as a plethysmograph and controls one or more phototherapy modules during the desired phase of the circulatory pulse cycle. The pulse oximeter as shown here may also contain the power source for the phototherapy modules as well as the microprocessor for control of the emitters, such as control microprocessor 430.

If a perfusion timing detector or pulse oximeter is used with one or more miniature phototherapy apparatuses, it or several of the miniature modules may be triggered by a single perfusion detector. Because of cost this perfusion timing detector may not be disposable or for single patient use.

Timing phototherapy so that it is not on continuously has the additional advantages of creating less heat buildup, and better utilizes battery power. These apparatuses could be made for single patient use, and this makes it easier to allow the infant to be treated at home. Battery power makes this easier. Use of single patient use hyperbilirubinemia apparatuses may allow earlier hospital discharge and may save on cost, especially in infants with mild hyperbilirubinemia.

The present disclosure thus teaches the method of timing the delivery of phototherapy to correlate to certain phases of the pulse cycle. Further the present disclosure teaches the method of incorporating a photoplethysmograph or pulse oximeter into a phototherapy apparatus, wherein at least some of the light required is provided by phototherapy emitters.

Thus, phototherapy apparatus 701 illustrated in FIG. 7A and FIG. 7C for example relates to an apparatus for phototherapy to target tissue of a subject that includes at least one light source, e.g., light sources 405a, 405b, 405c, that is configured to deliver phototherapy with a peak wavelength between 580 and 1350 nm,

at least one light sensor 785 that is configured to detect light passing through the target tissue and changes in the light passing through the target tissue and

at least one processor, e.g., a microprocessor housed in pulse oximeter 740, that is configured to do at least one of the following:

a. (1) measure changes in at least blood volume and/or light absorption of the blood passing through the target tissue; (2) enable correlation with respect to the subject, of the changes in the light passing through the target tissue with at least the timing of the pulse, and/or the pulse pressure, and/or the oxygen saturation of the blood, and/or the hemoglobin content of the blood, and/or the respiratory cycle; and (3) control the timing of delivery of phototherapy according a portion of the pulse cycle.

Referring specifically to FIGS. 7A and 7C for example, phototherapy apparatus 701 includes at least one of the following: (a) at least one light sensor 785 that is configured to detect data samples of light passing through target tissue and of changes in the light passing through the target tissue; (a) at least one processor, e.g., a microprocessor as housed within pulse oximeter 740 for example. The processor, e.g., the microprocessor housed within pulse oximeter 740, is capable of determining from the data samples, and with respect to a subject, at least the timing of the pulse, and/or the pulse pressure, and/or the respiratory cycle, and with respect to a subject and/or a target tissue, determining from the data samples at least one of the oxygen saturation of the blood and the hemoglobin content of the blood; and is capable of timing the delivery of phototherapy according to a predetermined phase of the pulse cycle.

### EMBODIMENTS OF THE APPARATUS

Several embodiments of phototherapy apparatuses are now disclosed or disclosed in further detail which are capable of timing and delivering phototherapy according to the methods discussed in the present disclosure.

One embodiment of the present disclosure is as a miniature phototherapy (PT) apparatus which is illustrated in FIG. 4. This apparatus may be configured for single patient use, and or single use “disposable”. FIG. 4A, FIG. 4B, and FIG. 4C show various perspective views of miniature phototherapy unit 400. This apparatus may be of variously sized, and for example may be about 2.5 cm wide, 4 cm long and 0.4 cm in depth in a certain embodiment, however these dimensions are in no way meant as a restriction to the size of the units.

Illustration FIG. 4A shows the front surface where in a certain embodiment multiple surface mounted L.E.D.s 405 are connected to substrate 440. The individual L.E.D.s may be of various individual peak wavelengths or may be combined wavelengths wherein a single L.E.D. may emit more than one narrow spectrum wavelength. In FIG. 4A, the L.E.D.s 405 are labeled 405a and 405b to illustrate one possible configuration where L.E.D.s of two different wavelengths are used. This is not meant to limit the number of L.E.D.s, the arrangement or the number of or types of emitters used in this present disclosure, but rather to illustrate one of many possible configurations. On the back side of the apparatus in FIG. 4B an electrical connector 415 is shown with a three electrodes 420 mounted on substrate 440. In other embodiments there may be two or more electrodes as required for the desired function of the apparatus. For example, if timing is coordinated between multiple units or modules, one or more electrical connections may be required for this function. The connector as shown is intended for a slide connector to fit into the slot for powering the apparatus. In this manner a single power source, such as a battery (not shown), may power single or multiple units or modules. The apparatus may also be configured to hold a battery such as a coin shaped battery 460 to power the unit as illustrated in FIG. 4D. In this embodiment battery compartment 455 may be used in place of electrical connector 415. In another embodiment electrical connectors 750 may be connected to a power supply and supply one or more PT units or modules 400 and 700. Such a power supply may be one or more batteries. Also shown is an embedded microprocessor 430. In FIG. 4C a profile of one embodiment of the apparatus is shown where the emitters are mounted to the substrate 440. A clear polymer 435 covers the surface mounted L.E.D.s and seals the electronics of the apparatus so that it can be cleaned, and so that it may be used against the skin. The front surface of the apparatus 410 comes into optical communication with the target tissue, allowing the light from the emitters to transmit to the target tissue. Also illustrated is a reflective surface 450 on the face of the apparatus which is disposed below the clear polymer to aid in reflecting light back to the tissue which may have been reflected and not absorbed by the tissue. In this example, the reflective surface 450 is shown with oval cutouts for the L.E.D. emitters 405. In FIG. 4C, small dotted arrows 470 illustrate the general direction of light emitted from the L.E.D. emitters 405, away from the emitters 405 and substrate 440, and towards the target tissue 10. The L.E.D.s 405 are shown with a wide beam angle used according to one embodiment of this disclosure. In another embodiment narrow beam angle emitters may be used. Some of the light emitted may be reflected from the surface of the polymer cover 435 and from the skin, e.g., target tissue 10, of the person or subject being treated for example. Reflective surface 450 acts to reflect light back towards the target tissue 10.

Thus, the phototherapy apparatus 400 may include reflective surface 450 that is configured to reflect light from the one or more emitters 405 towards the target tissue 10. The Phototherapy unit 400 is illustrated with an oval shape as per one embodiment of this disclosure. The small oval shape is illustrated as it has the advantages of lacking pointed edges and being adaptable to multiple areas where it may be used as a dressing for a wound. The phototherapy units may be used individually or multiple units may be used. When multiple units are used together they may have a single power source. It can be appreciated that, with reference to FIGS. 4A through 4E and FIGS. 7A, 7C, 7D and 7E, the present disclosure relates to an apparatus for delivering phototherapy, e.g., phototherapy apparatus 400 in FIGS. 4A through 4C or phototherapy apparatus 700 in FIGS. 7A and 7C. For example, phototherapy apparatus 400 includes at least one substrate 440 that is configured to enable mounting at least one light emitter, e.g., as shown in FIGS. 4A and 4C, at least one emitter 405 is mounted on one or more substrates 440, and is capable of emitting at least two peak wavelengths of light (for example 405a and 405b). An electronic circuit 430 is configured to control the timing of emission of the one or more emitters 405. The electronic circuit 430 is in electronic communication with the one or more emitters 405 and the apparatus 400 is configured as a dressing for optical communication enabling irradiation of a target tissue 10. As illustrated in FIG. 4E, in one embodiment, the phototherapy apparatus for delivering phototherapy may also be configured as phototherapy apparatus 400′ having at least one laser emitter 480 mounted on substrate 440. The clear polymer 435 may again be overlaid on the front surface 410 of this substrate 440. The electronic circuit 430 includes at least one processor 432, e.g., a microprocessor that is disposed internally within the electronic circuit 430. The processor 432 is configured to control temporal sequencing of emission of light by the one or more emitters 450 at least two different peak wavelengths.

FIG. 4E illustrates the upper face of a miniature phototherapy unit 400′ according to one embodiment of the present disclosure using two (2) laser diodes 480.

As defined herein, a dressing is an adjunct used for application to a wound in order to promote healing and/or prevent further harm, or applied to the body as a treatment for a condition, which is usually intended to remain in place for at least for several hours. As also defined herein, a dressing is designed to be in direct communication with the wound or tissue to be treated, which makes it different from a bandage, which is primarily used to hold a dressing in place, but does not itself have a medicinal property. Thus, various embodiments of this disclosure use phototherapy units and modules as dressings for treatment.

Returning to FIG. 7A, and FIG. 7C in more detail, FIG. 7A shows the front side of a miniature phototherapy module 700 which directs the light from the emitters towards the target tissue. FIG. 7C shows the back side of module 700 with socket 705 in electrical connection with pulse oximeter unit 740. Connector 705 is mounted on substrate 780 and shown with four electrodes 710, but may the connector have more or less electrodes according to the requirements of various configurations of the present disclosure. Also shown is microprocessor 430. In other embodiments of the present disclosure microprocessor 430 may be separate from photo therapy module 700, but in electrical communication with emitters 405. On the front side of miniature phototherapy module 700 L.E.D.s 405a, 405b, 405c are shown, as well as photosensor 785, mounted on substrate 780. L.E.D.s 405a, 405b, and 405c are shown to illustrate various L.E.D. emitters which may be used, but are not intended to restrict the population or configuration of emitters used in this disclosure.

FIG. 7C also shows multiple connectors 750 which may slide into and connect to the socket 705 on the phototherapy modules. Electrical contacts 715 make electronic communication with electrodes 710. Photosensor 785 may be used for plethysmography or pulse oximetry under various embodiments of the present disclosure. Alternatively one or more L.E.D.s may be used as photosensors. The pulse oximeter may be powered by batteries, and or an external power source.

FIG. 7D illustrates the back side of terminals 750. A branching ribbon connector 755 connects the terminals showing connections but not shown to scale. A terminal 760 on the ribbon connector 755 plugs into a slit socket 770 on the back side of terminal 750. A cutaway view is shown 775. In FIG. 7 bifid branching ribbon connectors are shown, but the ribbon connectors may also be single, in order to form a daisy chain of photo therapy modules, or may be multiple with 3 or more branches for each terminal group. In another embodiment these ribbon connectors are substituted with wires. Also shown in FIG. 7D is battery compartment 790.

FIG. 9 illustrates how multiple miniature phototherapy modules 700 may be “tiled” to treat an area. The pulse oximeter, timing microprocessor and power unit which may include batteries, are shown housed in 905. Electrical connectors 915 connect the components. Also shown is a photosensor 910 portion of pulse oximeter probe for use in oximetry measurements when the light source is integrated as part of the phototherapy apparatus. This photosensor may be situated to detect transmitted light depending on the location of the treatment area, or may be configured for detecting reflected light, such as may be reflected by bone. Reflected light is usually sufficient for photoplethysmography. Alternately a photosensor 780 may be configured into the miniature phototherapy module 700 as illustrated in FIG. 7A.

Phototherapy may be applied, for example, to the leg 950 at the area of the lateral ankle, a site which may be affected by arterial insufficiency for example. FIG. 9 illustrates phototherapy units 700 held in place and covered by a bandage 920. Also shown is power source 905 connected to the units by wires 915.

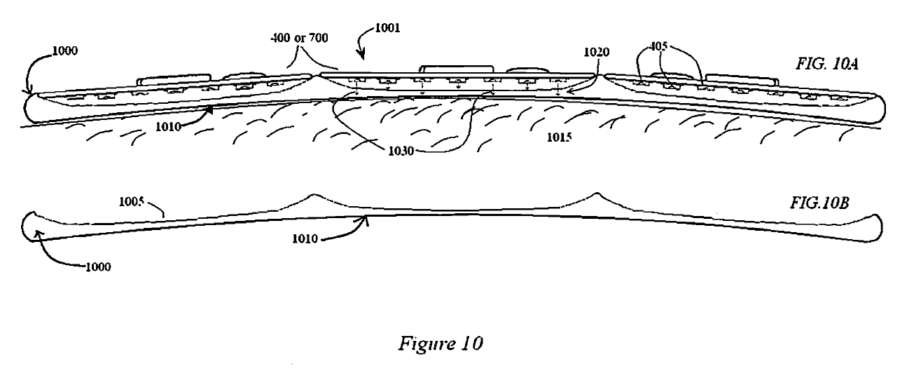

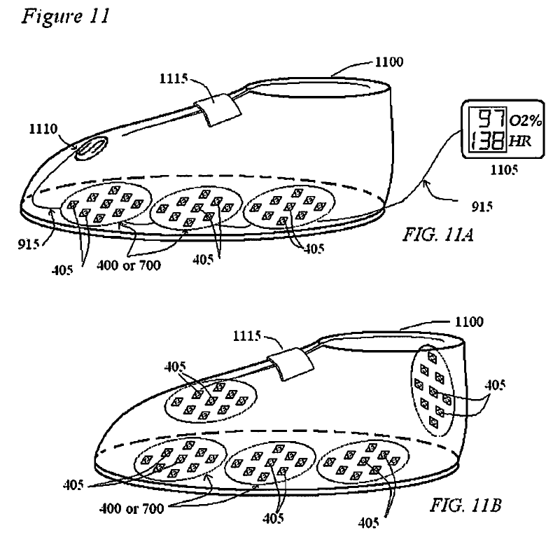

FIG. 10A shows a cross section of a phototherapy apparatus 1001 in which phototherapy units or modules 400 and 700 embedded in a clear or translucent wound dressing 1000 which transmits the light frequencies delivered by the phototherapy units and modules 400 and 700 through the translucent dressing 1000 to the tissue. Small arrows 1020 show light being emitted to the skin 1015. This dressing may be for example as a hydrogel or hydrocolloid material and dressing may have antimicrobial and wound healing properties. This translucent dressing 1000 is positioned between the subjects tissue and the PT units and modules so they are not in direct contact with the skin 1015 or with the wound. The wound dressing may be made to conform to the shape of the units, as shown for example in FIGS. 10A and 10B. Shown for example, a depression 1005 for units and modules 400 and 700 and may have depressions 1005. The dressing may be made with adhesives, or have adhesives applied to it so that the PT units or modules, for example 400 and 700 stay in place on the material, and may be made so that the patient surface 1010 adheres to the skin or tissue 1015 that is the intended area for exposure. The light 1030 emitted from the phototherapy units and modules 400 and/or 700 is transmitted through the clear dressing 1000 to the target tissue 1015. FIG. 10B shows one embodiment of the wound dressing material without the miniature phototherapy units, with depressions 1005 formed for fitting the units. Thus in one configuration according to this disclosure, one or more miniature phototherapy units or modules may be affixed to a translucent dressing on one side and be in communication with the tissue to be treated on the other side. In one embodiment of this disclosure the translucent dressing may have healing properties, and antimicrobial properties.

Thus it can be appreciated that phototherapy apparatus 1001 includes a translucent wound dressing 1000 that is configured wherein the light 1030 of the one or more emitters 405 is directed through the translucent dressing 1000.

If a bandage is applied with excessive pressure it can potentially be damaging to the tissue. A hydrogel or other wound dressing which is transparent to the light frequencies used by the PT unit or module can be flexible, and thus conform to the shape of the area being treated. The adhesive allows a bandage which does not require pressure in order to hold the phototherapy units in place. This is intended to help decrease the use of pressure on the treatment area.

Miniature phototherapy units and modules may be configured for use within a bandage, or as apparel which may be worn. In one embodiment the phototherapy apparatus 1100 may be configured as a bootie as shown in FIGS. 11A and 11B. Hyperbilirubinemia is a common problem in preterm infants. Because melanin is a strong interfering substance for photo-conversion of bilirubin, target areas with little pigmentation such as the soles of the feet may be appropriate especially in darkly pigmented full term infants.

In one embodiment of this apparatus the phototherapy units and/or modules may be structured into booties that may be worn, as illustrated in FIG. 11A and FIG. 11B. One advantage of this is that the skin of the palms and the soles have less pigmentation and thus less of the therapeutic EMR is absorption by non-target molecules. This is especially important for darkly pigmented individuals. The hands and feet have high blood flow and thus are effective areas for phototherapy of blood born molecules such as bilirubin. For infants this isolates the light from the face where blue light might pose risk to the eyes. The garments could be made to attenuate or block some or most of the light. They may also be made to preferentially allow red light to pass for example, so that personnel could see that the phototherapy was active. In a similar configuration a Red/IR phototherapy bootie may be configure for healing the feet in patients with compromised circulation, such as might be found in patients with diabetes. In other configurations of this apparatus the phototherapy units and modules may be structured into wraps, clothing, bandages or other forms that may be worn.

In FIG. 11A, the bootie 1100 is in electrical communication with a pulse oximeter 1105 that may or may not control the pulse cycle of the PT apparatus. Connectors 915 allow electrical communication between the electrical components. Above the toe a photoreceptor 1110 (also herein referred to as a photosensor) is shown for pulse oximetry and/or plethysmography which may be used to collect clinical data, and/or to control the phototherapy apparatus. Alternatively, a photosensor within a phototherapy apparatus such as photosensor 780 in module 700, or photosensor 1415 in phototherapy module 1400 may be used for this purpose.

Thus, referring to FIG. 11A, the present disclosure teaches a method of phototherapy, wherein a desired phase of the circulatory cycle of a subject is determined by detecting, via the pulse oximeter 1105, a variance in transmission of light through tissue of the subject.

Along the inside of the bootie are shown PT modules 400 or 700. In FIG. 11A they are directed towards the sole of the foot where pigment levels are low, while in FIG. 11B one is shown along the ankle. If no pulse oximetry apparatus is used, external wiring may not be needed if sufficient power can be obtained from one or more batteries included in the bootie supplying several PT modules, or in another embodiment each phototherapy units may be electrically independent of each other, as illustrated in FIG. 11B. In one embodiment of this disclosure the power for these independent phototherapy units would a battery as shown in FIG. 4D. In another embodiment, the power requirements for the units may be supplied by a battery though a connector 415 such as illustrate in FIG. 4A. In both images a closure strap 1115 such as Velcro™ is used close the bootie, however multiple other methods for fitting may be used.

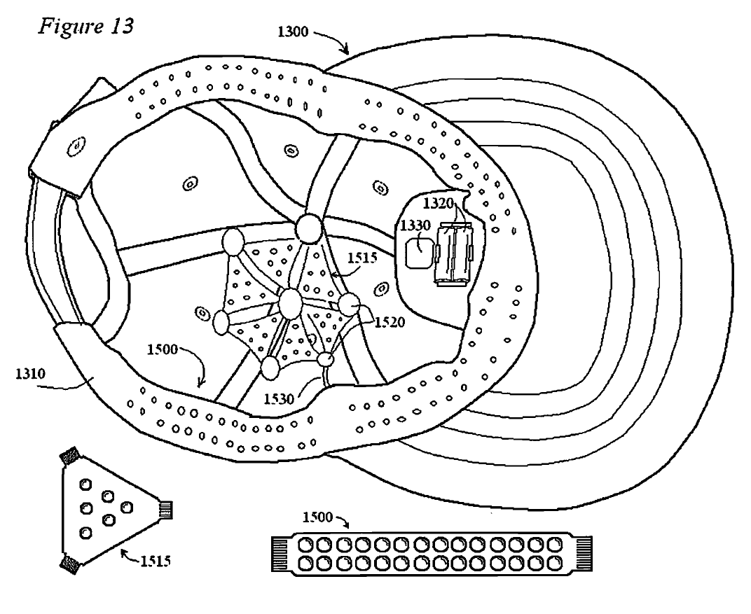

The phototherapy technology as described in the above booty may be incorporated into bandages or clothing including mittens, leg wraps, arm wraps, body wraps, head bonnet, skin dressing, diaper or other clothing. In particular, with reference to FIGS. 11A, 11B and FIG. 13 for example, at least a portion of the phototherapy apparatus, e.g., phototherapy modules 400 or 700 in FIGS. 11A, 11B or phototherapy modules 1515 in FIG. 13, is configured to be wearable by a subject and specifically is configured as a piece of apparel 1100 (a bootie in FIG. 13) and a bandage 920 in FIG. 9.

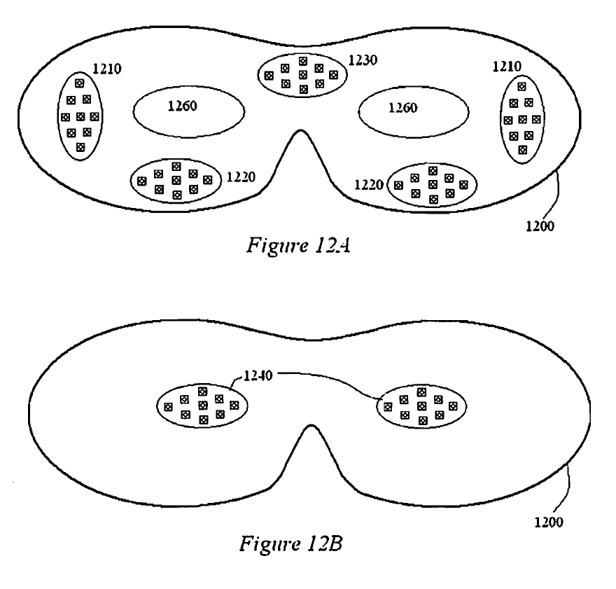

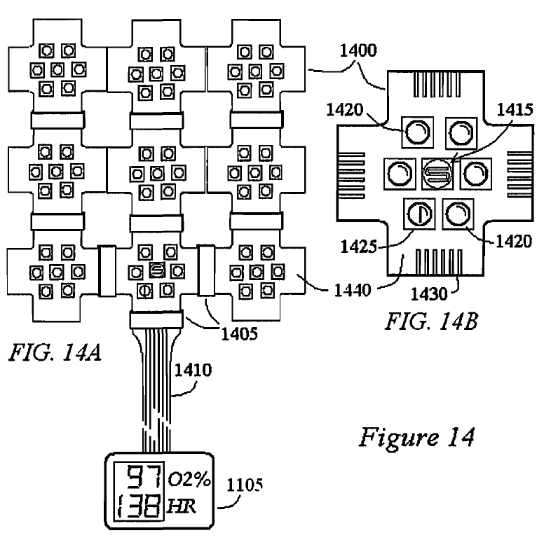

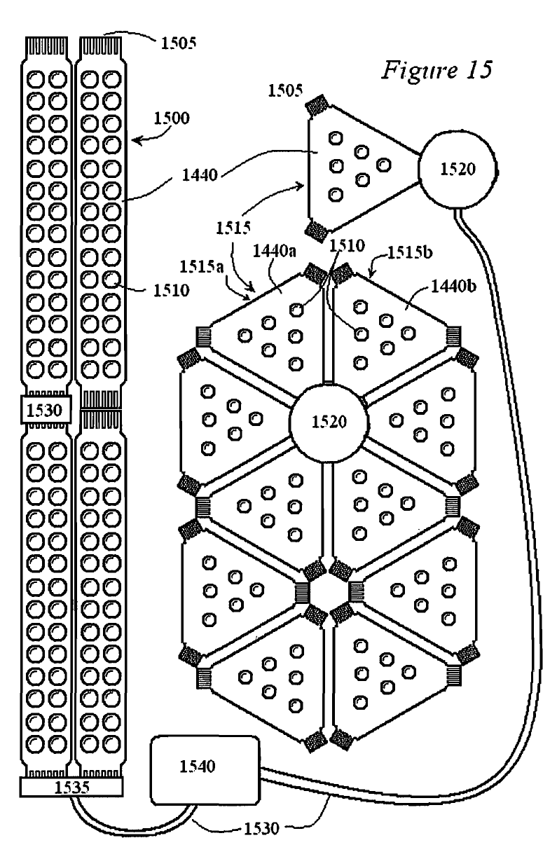

The phototherapy units and modules may be of various shapes and sizes. FIG. 14 and FIG. 15 show other exemplary conformations for phototherapy modules. These modules may have various connection sites that allow them to be connected to form a chain or grid depending on the geometry of the area to be exposed. In other embodiments of this present disclosure, emitters such as L.E.D.s 1420 and 1520 may be mounted on flexible printed circuit substrate 1440 as illustrated in FIGS. 11 and 12.

Multiple single units or modules may be used to tiled and provide phototherapy over a broader area, as illustrated in FIGS. 7, 11 and 12. The small size of the units and modules allow then to be utilized like tiles which can conform to the contour of the area being treated. They may be configured to attach to and be held in place by a bandage. For example several units or modules may be attached to a bandage for treatment of conditions such as diabetic neuropathy of the lower limb. As part of its design it may be adapted to consume less power than traditional phototherapy devices, and may be powered by one or more batteries, depending on voltage requirements, the size of the area being treated, and the duration of the treatment. Such a apparatus may be worn while the patient is ambulatory.

FIG. 12A illustrates a phototherapy mask 1200 which may be worn to apply phototherapy treatment. It may be used while sleeping for example. The inside of the mask is shown. In this illustration several miniature PT units and modules 400 are 700 are utilized. FIG. 12B illustrates a phototherapy mask configured to deliver photo therapy to the eyes. In the embodiments illustrated in FIGS. 12A and 12B the power source may be batteries as illustrated in FIG. 4D. In another embodiment according to this disclosure the phototherapy modules may be connected to one or more batteries. Example of this are illustrated as illustrated in FIG. 7D and FIG. 9.

In one embodiment the apparatus may take the form of a mask so that it can be easily worn for treatment around the eyes as illustrated in FIG. 12A. Dark circles and bags under the eye are a common cosmetic problem. Infraorbital discoloration, and puffyness of the skin are thought to have a variety of causes, including dermal melanin deposition, postinflammatory pigmentation, superficial dermal blood vessels, and deposition of fat. The present disclosure teaches the method of using phototherapy for treating dark circles and bags, discoloration or wrinkles below and around the eyes. The lateral PT units 1210 as shown may be used for treatment of smile lines or wrinkles. The inferior PT units 1220 may be used for treatment of discoloration, swelling (bags) or wrinkles under the eyes. PT unit 1230 may be positioned for treatment of wrinkling of the area between the eye brows. The face mask may be custom designed to match specific face geometry with an array of strategically positioned light sources to match local skin lesions with specific wavelengths. In an alternative embodiment emitters such as surface mounted L.E.D.s, may be mounted on a flexible printed circuit configured to conform to the face, or to other areas of the body. The mask may optionally have apertures for the eyes 1260 so that it may be worn while awake. The mask may be made with one or more pockets integrated to the mask which can hold inserts to cover the eyes for providing darkness.

FIG. 12B illustrates two PT units 1240 configure to direct phototherapy to the eyes. PT units may be configured for treatment of retinal conditions including conditions of the retina pigmented epithelium, and may be configured with light emitters which transmit light through the eyelid so that therapy can take place during rest. Phototherapy modules may also be used in these configurations. Phototherapy might be useful for treatment of retinal diseases, such as retinitis pigmentosa, macular degeneration, retinal dystrophies, glaucoma, or diabetic retinopathy.

FIG. 13 illustrates an apparatus according to one embodiment of this disclosure for providing phototherapy to the head and scalp. The present disclosure teaches the embodiment of an phototherapy apparatus integrated into headwear to facilitate delivery of phototherapy to the head. In FIG. 13 the inside view of a baseball cap 1300 is illustrated. A sweatband 1310 is shown which is made of a translucent material to allows light to pass towards the scalp from a miniature phototherapy modules 1500 in which L.E.D.s are mounted on a flexible substrate. Also illustrated in this embodiment are several exemplary phototherapy modules 1515 which are positioned to irradiate the scalp. Six triangular modules 1515 are shown, however more or fewer may be utilized, and PT units or modules may line the inner surface of the cap. Batteries 1320 and microprocessor 1330 are illustrated according to one embodiment of this disclosure. Electrical connector 1530 is also shown. FIG. 13 shows a phototherapy apparatus configured as a baseball cap, however it may also be configured as a bonnet, hat, scarf or other form of headwear.

The present disclosure teaches that phototherapy may be used to treat hair loss. Transcranial phototherapy may also be done to treat intracranial lesions. For example, it has been demonstrated that animals with induced stroke like lesions recover better if they receive transcranial phototherapy. Various embodiments according to the present disclosure may be used to treat superficial conditions such as skin lesions and hair loss, to improve healing of the tissues, and to treat intracranial conditions including memory loss and other conditions of the brain amenable to phototherapy.

The present disclosure shows miniature phototherapy units and modules used modularly for delivery of phototherapy to various areas. Other configurations for miniature phototherapy modules are illustrated in FIG. 14 and FIG. 15.

FIGS. 14 and 15 show alternative embodiments to the phototherapy modules which may be linked together to give phototherapy coverage for different sized areas depending on the size of the area to be treated. FIG. 14 illustrates L.E.D.s mounted on flexible printed circuit material. The modules 1400 are linked together physically and electrically by connectors 1405. By not using unnecessary connectors, the array of modules are given more flexibility and thus may conform better to the surface being treated. A ribbon connector 1410 allows connection to a power supply and microprocessor. FIG. 14B shows a detailed view with a photosensor 1415 and a dedicated pulse oximetry L.E.D. 1425 which is meant to indicate an LED with two wavelength emitters, as well at the therapeutic L.E.D.s 1420. Also illustrated in FIG. 14A only a single module in the grouping shows the photosensor 1415 and pulse oximetry L.E.D. 1420. Electrodes are illustrated 1430. A pulse oximetry unit 1105 is shown which may be used with these PT modules in one embodiment of this invention. In another embodiment only plethysmography is utilized.

FIG. 15 shows two further embodiments of phototherapy modules with emitters mounted on flexible PCB material. In FIG. 15 a linear embodiment 1500 is shown which might be useful for longer lesions, or for wrapping a limb. There are electrodes 1505 on the ends for connecting to the microprocessor and power supply for the apparatus. Rows of L.E.D.s 1510 are illustrated. A connector 1530 to link modules is shown. PT modules may be placed in an elastic wrap to stay in position for treat of a joint such as the knee or elbow. Connector 1235 allows an external power supply, and/or control unit 1540, via connector wire 1530.

In view of the previous discussion of phototherapy apparatus 400 with respect to FIGS. 4A-4C, it can be appreciated that, with references to FIGS. 14A-14B and 15, FIGS. 12A and 12B and 13, and FIGS. 9 and 11A for example, the present disclosure relates to an apparatus for delivering phototherapy, e.g. phototherapy apparatus 1400 in FIGS. 14A-14B, phototherapy apparatuses 1500 and 1515 in FIG. 15, phototherapy apparatus 1200 in FIGS. 12A and 12B, phototherapy apparatus or modules 1515 in FIG. 13, having at least one substrate, e.g., substrate 1440 that is at least a first substrate 1440a and a second substrate 1440b (see FIG. 15). At least a first emitter 1510 is emitter mounted on the first substrate 1440a and at least a second emitter 1510 is mounted on the second substrate 1440b. The first substrate 1440a and the one or more first emitters 1510 define a first modular phototherapy apparatus 1515a and the second substrate 1440b and the one or more second emitters 1510 define a second modular phototherapy apparatus 1515b. The first and second modular phototherapy apparatuses 1510a and 1510b, respectively, are at least physically connected to at least one another, e.g., via a connector 1520, and/or mounted on and/or within a common structure, e.g., the baseball cap 1300, and/or in electric communication with each other, e.g, via electrical connector wire 1530.

A roughly triangular embodiment 1515 is shown in FIG. 15 which may be useful to cover a larger area, or for example to tread the scalp area, where the apparatus would need to conform to a bowl shape. In FIG. 15 a connector 1520 is shown which allows connection to several phototherapy modules 1515 at one time and allows much flexibility to the shape, size and area to be treated. Electrical connector wire 1530 connects between connector 1520 and the power supply and/or control unit 1450.

The illustrations in FIGS. 4, 7, 14 and 15 show embodiments with L.E.D. lights. L.E.D. light sources have several advantages including narrow spectral width, good energy efficiency, long life, lack of mercury used in there production and low heat production, and fast cycling (turning on and off). Some currently used light sources for treating neonatal jaundice must be replaced after as little as a thousand hours as there emission fades, while L.E.D. lights have long lives and are inexpensive. Energy efficiency is important for ambulatory products which run on batteries, photovoltaic or other mobile energy sources. Specific narrow spectrum lights have advantages over broad spectrum light sources. Broad spectrum sources such as halogen bulbs and fluorescent lamps may include U.V. light (less than about 400 nm) which may be injurious to infant and to caretakers. Even blue light holds risk for retinal damage. Specific narrow spectrum sources will excite their target molecules more efficiently at lower light levels, and produce less heat.

The present disclosure also teaches the use of long duration (hours) cycling sequential wavelength, and timed (to phase of pulse) phototherapy. It may be designed to be used for extended periods of hours or even days rather than minutes. Phototherapy is typically used with a constant light source for a short session, for example, a 15 minute session of high intensity light. The present disclosure teaches the use of multiple pulses of light, delivered over an extended period. Thus in place of a 15 minute session once a day or even less frequently, the present disclosure teaches the use of pulses of light over several hours or delivered continuously, for example overnight or may be worn for days.

The present disclosure teaches the use of ambulatory phototherapy, where the user may wear a phototherapy apparatus with a self contained power supply.

In particular, FIG. 9 illustrates

at least one power source 905 and FIG. 13 illustrates at least one power source 1320, for example, wherein the one or more power sources 905 and 1320 are configured to provide power to the apparatus, e.g. to phototherapy modules 700 in FIG. 9 or to phototherapy modules 1515 in FIG. 13, to effect the emission of the light. The power sources 905 and 1320, for example, may be a power source having sufficient capacity to power the emitters 405a, 405b, 405c (see FIG. 7A) or emitters 1510 (see FIG. 15) for at least one hour, and/or a battery, e.g., battery 1320 in FIG. 13, and/or a power source, e.g., power source 705 in FIG. 7C, configured for ambulatory use.

One method for the delivery of phototherapy as taught here is the use of a wearable apparatus which can be applied to or worn by the patient. Although U.S. Pat. No. 6,866,687 describes a light bandage, it is not intended for continuous or ambulatory use, and does not have a self contained power supply. It would not fit under typical clothing. The current invention is intended to be produced at low cost, so that it would be inexpensive enough for single patient use, or disposable.

The present disclosure illustrates multiple embodiments for phototherapy which use a miniature and modular apparatuses. These incorporate L.E.D. emitters, which may be of multiple narrow wavelength emitters. These apparatuses may include photosensors, or use L.E.D.s as photo-sensors for photoplethysmography. These apparatuses may include or be controlled by microprocessors which allow for rapid sequential timing of the emitters. These apparatuses may include or be controlled by microprocessors which allow for timing according to a portion of the circulatory pulse cycle. These apparatuses may be integrated into wearable embodiments such as booties, gloves, masks, bandages, caps or other wearable configurations. These apparatuses may be integrated to a transparent dressing configured to hold the phototherapy units and modules in place, and direct the phototherapy to the target tissue. This dressing may be a hydrogel or similar material, and may have healing or anti-infective properties of its own, and may allow the placement of this dressing directly to a wound area.

As described herein, the phototherapy apparatuses are applied to emit light to a subject, or target tissue thereof, wherein the subject, or target tissue thereof, is a human being, an animal, an insect or a plant or a biological substance such as blood, saliva or other similar fluid. The target tissue may be either internal to a subject or external to a subject. In particular, the biological substance may be either internal to a subject or external to a subject, e.g., outside of the body. Those skilled in the art will recognize that the phototherapy apparatus described herein may also be applied to emit light to influence or affect or effect the outcome or direction of a chemical reaction or a physical process.

Many modifications and other embodiments of the present disclosures set forth herein will come to mind to one skilled in the art to which these disclosures pertain having the benefit of the teachings presented in the foregoing descriptions and the associated drawings. Therefore, it is to be understood that these disclosures are not to be limited to the specific embodiments disclosed and that modifications and other embodiments are intended to be included within the scope of the appended claims. For example, although the embodiment shown in FIG. 3 shows an emitter with a wavelength of 830 nm followed sequentially by one or more emitters with wavelengths of 670 and 606 nm, the emitters following the first emitter may be used simultaneously, and although L.E.D.s may be utilized in this description it is not meant to limit the embodiments of the present disclosure to their use. Additionally those skilled in the art will know that L.E.D. emitters may be configured to emit more than a single peak wavelength, and that bicolor or multicolor L.E.D. emitters may be used in this apparatus. For example, a RGB L.E.D. may be used in the photo-conversion of bilirubin to lumirubin using the blue element and then the green and red elements. Although specific terms are employed herein, they are used in a generic and descriptive sense only and not for purposes of limitation.

Although phototherapy for the conversion of bilirubin is principally used for infants, it may also be used in older patients, and may be used to treat jaundice, and be used locally to treat hematoma, thus this disclosure does not limit phototherapy for conversion of bilirubin to neonatal hyperbilirubinemia.

This list of embodiments enumerated above is not intended to limit the scope of the present disclosure described herein, but rather to highlight the scope of the disclosure. It is intended that the broadest interpretation of this disclosure is claimed by this disclosure.

What is claimed is:

1. An apparatus for delivering phototherapy comprising;
at least one substrate configured to enable mounting at least one light emitter;
at least one emitter mounted on the at least one substrate, and capable of emitting at least two peak wavelengths of light; and
an electronic circuit configured to control the timing of emission of the at least one emitter,
wherein the electronic circuit is in electronic communication with the at least one emitter, and
wherein the apparatus is configured as a dressing for optical communication enabling irradiation of a target tissue.

2. The apparatus for delivering phototherapy according to claim 1 wherein the at least one light emitter is at least one of a laser emitter and a light emitting diode.

3. The apparatus for delivering phototherapy according to claim 1, wherein the electronic circuit includes at least one processor and wherein the at least one processor is configured to control temporal sequencing of emission of light by the at least one emitter at least two different peak wavelengths.

4. The apparatus for delivering phototherapy according to claim 1, wherein the at least one emitter is configured with at least one of:
(a) a blue emitter and one of a phosphor and a scintillator, the phosphor and the scintillator emitting light at least one peak wavelength longer than 500 nanometers (nm);
(b) a light emitting diode that emits white light; and
(c) at least one light emitter configured to deliver a pulse of irradiation to the tissue with a duration of less than a second.

5. The apparatus for delivering phototherapy according to claim 1,
wherein the at least one substrate comprises at least a first and a second substrate; and
the at least one emitter comprises at least a first emitter mounted on the first substrate and at least a second emitter mounted on the second substrate,
wherein the first substrate and the at least first emitter define a first modular phototherapy apparatus and the second substrate and the at least second emitter define a second modular phototherapy apparatus, and
wherein the first and second modular phototherapy apparatuses are at least one of:
a. physically connected to at least one another;
b. mounted at least one of on and within a common structure; and
c. in electric communication with each other.

6. The apparatus for delivering phototherapy according to claim 1, further comprising
at least one power source,
wherein the at least one power source is configured to provide power to the apparatus to effect the emission of the light, and
wherein the at least one power source is at least one of:
(a) a power source having sufficient capacity to power the emitters for at least one hour;
(b) a battery; and
(c) a power source configured for ambulatory use.

7. The apparatus for delivering phototherapy according to claim 1, further comprising a reflective surface configured to reflect light from the at least one emitter towards the target tissue.

8. The apparatus for delivering phototherapy according to claim 1,
wherein at least a portion of the apparatus is configured to be wearable by a subject and wherein the at least a portion of the apparatus that is configured to be wearable by a subject is configured as at least one of a piece of apparel, a dressing and a bandage.

9. The apparatus for delivering phototherapy according to claim 1 further comprising a translucent dressing configured wherein the light of the at least one emitter is directed through the translucent dressing.

10. The apparatus for delivering phototherapy according to claim 1, further comprising at least one of:
at least one light sensor configured to detect data samples of light passing through the target tissue and of changes in the light passing through the target tissue; and
at least one processor:
(a) capable of determining from the data samples, and with respect to a subject, at least one of the timing of the pulse, the pulse pressure, and the respiratory cycle, and with respect to at least one of a subject and a target tissue, determining from the data samples at least one of the oxygen saturation of the blood and the hemoglobin content of the blood; and
(b) capable of timing the delivery of phototherapy according to a predetermined phase of the pulse cycle.

11. The apparatus for delivering phototherapy according to claim 3, wherein at least one of:
(1) wherein the at least one processor at least one of:
(a) is configured to control the at least one emitter to deliver at least a first pulse and at least a second pulse of light for irradiation to the target tissue;
(b) is configured to control the at least one emitter to repeat the at least first pulse of light and at least second pulse of light as pulse sets; and
(c) is configured to create a delay between pulse sets;
and (2) wherein the at least one emitter at least one of:
(a) is a blue emitter with a wavelength ranging between about 450 and about 500 nanometers (nm) and wherein light emitted from the blue emitter is delivered with a first pulse, and includes at least one second emitter with a wavelength ranging between about 500 and about 700 nanometers (nm) and wherein light emitted from the at least one second emitter is delivered with a second pulse in the pulse set; and
(b) is an emitter with a wavelength ranging between about 800 and about 900 nanometers (nm) and wherein light emitted from the at least one emitter is delivered with a first pulse, and includes at least one second emitter with a wavelength ranging between about 600 and about 700 nanometers (nm) and wherein light emitted from the at least one second emitter is delivered with a second pulse in the pulse set.

12. An apparatus for phototherapy to target tissue of a subject, comprising
at least one light source configured to deliver phototherapy with a peak wavelength between 580 and 1350 nm;
at least one light sensor configured to detect light passing through the target tissue and changes in the light passing through the target tissue; and
at least one processor configured to at least one of:
(a) measure changes in at least one of blood volume and light absorption of the blood passing through the target tissue;
(b) enable correlation with respect to the subject of the changes in the light passing through the target tissue with at least one of the timing of the pulse, the pulse pressure, the oxygen saturation of the blood, the hemoglobin content of the blood, and the respiratory cycle; and
(c) control timing of delivery of phototherapy according a portion of the pulse cycle.

13. A method of applying phototherapy to a subject, comprising the steps of:
providing at least one light emitter;
delivering a first pulse of light to the target tissue of a subject from the at least one emitter with a peak wavelength of light; and
delivering at least a second pulse of light to the target tissue of the subject from the at least one emitter wherein the at least one emitter provides at least one peak wavelength of light that is different from the peak wavelength of the first pulse of light,
wherein the steps of delivering a first pulse of light and of delivering a second pulse of light define a method of delivering a series of pulse sets of light, and
wherein the first pulse of light and the second pulse of light define a pulse set of light.

14. The method according to claim 13, wherein the at least one emitter is at least one of a light emitting diode and a laser.

15. The method according to claim 13, wherein at least one of:
a. the step of delivering of the at least second pulse of light occurs at a time period of less than about one second after the step of delivering the first pulse of light;
b. the step of delivering of the at least a second pulse set of light occurs at a time period of less than about one minute after the step of delivering the first set of pulses;
c. the step of delivering the at least a second pulse set occurs at a time period of less than about one second after the step of delivering the first set of pulses; and
d. wherein the method of delivering a series of pulse sets of light includes the step of delivering at least three pulse sets over a time period greater than about one hour.

16. The method according to claim 13, wherein at least one of:
(a) the first pulse of light in the pulse set has a peak wavelength between about 450 nanometers and about 500 nanometers;
(b) the at least a second pulse of light has a peak wavelength between about 500 to about 700 nanometers (nm);
(c) the at least a second pulse of light has a peak wavelength between about 565 to about 700 nm; and
(d) the phototherapy is applied for treatment of at least one of hyperbilirubinemia, jaundice, hematoma and bruising.

17. The method according to claim 13, wherein at least one of:
(a) wherein the pulse set comprises at least:
a first pulse of light having a peak wavelength between about 800 nanometers and about 900 nanometers, and
wherein the at least second pulse of light has at least one peak wavelength between about 600 nanometers and about 700 nanometers; and
(b) the method of applying phototherapy is applied for treatment of at least one of injury, tissue degeneration, tissue discoloration, and hair loss.

18. A method for phototherapy for a subject comprising the steps of:
providing at least one light source emitting light with a peak wavelength less than about 500 nanometers;
providing at least a second light source emitting light with at least one peak wavelength ranging between about 565 nanometers and about 700 nanometers (nm);
delivering the light with a peak wavelength of less than about 500 nanometers to tissue of the subject for a time period of greater than about one hour; and
at least partially concurrently delivering the light with a peak wavelength ranging between about 565 and about 700 nanometers for a time period of greater than about one hour.

19. The method for phototherapy for a subject according to claim 18, wherein the phototherapy is for a subject with at least one of hyperbilirubinemia, jaundice, hematoma and bruising.

20. A method of timing delivery of phototherapy to a subject comprising the steps of:
measuring at least one phase of the circulatory cycle of the subject;
identifying a desired phase of the at least one phase of the circulatory cycle of the subject wherein the desired phase is beneficial for delivery of phototherapy to the subject; and
delivering phototherapy to the subject during at least a portion of the desired phase of the circulatory cycle of the subject.

21. The method according to claim 20, wherein the desired phase of the circulatory cycle is determined by detecting a variance in transmission of light through tissue of the subject.

22. A method of phototherapy treatment comprising the steps of:
providing at least one light emitter, and irradiating target tissue of a subject with light from the at least one emitter for sufficient time to give therapeutic effect, wherein the at least one emitter is at least one of:
(a) a blue emitter emitting light with a peak wavelength less than 500 nanometers (nm), the light emitted from the blue emitter being at least a portion of the light irradiating the tissue from the at least one emitter, the blue emitter coupled with one of a phosphor and a scintillator, the phosphor and the scintillator emitting light at least one peak wavelength longer than 500 nanometers (nm), the light from the one of a phosphor and a scintillator being at least a portion of the light irradiating the tissue from the at least one emitter;
(b) a light emitting diode emitting white light, the white light emitted from the light emitting diode being at least a portion of the light irradiating the tissue from the at least one emitter;
(c) configured to emit light cycling in intensity at a rate of at least one cycle per second, the light from the at least one emitter cycling in intensity being at least a portion of the light irradiating the tissue from the at least one emitter; and
(d) configured to emit light delivering a pulse of irradiation with a duration of less than one second, the light from the at least one emitter delivering a pulse of irradiation with a duration of less than one second being at least a portion of the light irradiating the tissue from the at least one emitter.

23. The method according to claim 22, wherein the subject has at least one of hyperbilirubinemia and jaundice.
